SEQUENCE LISTING (39 sequences)

SEQ ID NO: 1 - DNA, 21 bp, Artificial Sequence
  PCR primer
ccctgtgggttttacacttaa

SEQ ID NO: 2 - DNA, 19 bp, Artificial Sequence
  PCR primer
acgattgtgcatcagctga

SEQ ID NO: 3 - DNA, 22 bp, Artificial Sequence
  PCR primer
ggggaacttctcctgctagaat

SEQ ID NO: 4 - DNA, 21 bp, Artificial Sequence
  PCR primer
agacattttgctctcaagctg

SEQ ID NO: 5 - DNA, 21 bp, Artificial Sequence
  PCR primer
ctcggatccatctcactgcaa

SEQ ID NO: 6 - DNA, 21 bp, Artificial Sequence
  PCR primer
tgcaacaacatcatagagccg

SEQ ID NO: 7 - DNA, 22 bp, Artificial Sequence
  ATCCTAAAGGATTTTGTGACTT
atcctaaaggattttgtgactt

SEQ ID NO: 8 - DNA, 18 bp, Artificial Sequence
  PCR primer
tgcacttacaccgcaaac

SEQ ID NO: 9 - DNA, 49 bp, Artificial Sequence
  PCR primer
tgtttttaagtgtaaaaccacaggaaggtaagtatgtacaaatacctac

SEQ ID NO: 10 - DNA, 46 bp, Artificial Sequence
  PCR primer
gttatggctgtagttgtatcaactgtttaaaaacgattgtgcatca

SEQ ID NO: 11 - DNA, 20 bp, Artificial Sequence
  PCR primer
acgtagtcgcaacagttcaa

SEQ ID NO: 12 - DNA, 19 bp, Artificial Sequence
  PCR primer
tctgccgaaagcttgtgtt

SEQ ID NO: 13 - DNA, 38 bp, Artificial Sequence
  PCR primer
atcaccgccattgccagcccaactccaggcagcagtag

SEQ ID NO: 14 - DNA, 42 bp, Artificial Sequence
  PCR primer
gccaacaacaacaaggccaaactagtacgtttttgccgaggc

SEQ ID NO: 15 - RNA, 43 nt, Artificial Sequence
  CRISPR gRNA
uaauuucuacucuuguagaucacauaccgcagacgguacagac

SEQ ID NO: 16 - RNA, 41 nt, Artificial Sequence
  CRISPR gRNA
uaauuucuacucuuguagaucugcugcuugacagauugaac

SEQ ID NO: 17 - RNA, 41 nt, Artificial Sequence; identical to SEQ ID NO: 16
  CRISPR gRNa

SEQ ID NO: 18 - RNA, 42 nt, Artificial Sequence
  CRISPR gRNA
uaauuucuacucuuguagauagagcaacuucuucaagggccc

SEQ ID NO: 19 - DNA, 12 bp, Artificial Sequence
  CRISPR gRNA
tttttttttttt

SEQ ID NO: 20 - DNA, 24 bp, Artificial Sequence
  gRNA target
tgtaccgtctgcggtatgtggaaa

SEQ ID NO: 21 - DNA, 24 bp, Artificial Sequence
  DNA target of gRNA
acatggcagacgccatacaccttt

SEQ ID NO: 22 - DNA, 24 bp, Artificial Sequence
  gRNA target
tttgctgctgcttgacagattgaa

SEQ ID NO: 23 - DNA, 24 bp, Artificial Sequence
  gRNA target
aaacgacgacgaactgtctaactt

SEQ ID NO: 24 - DNA, 24 bp, Artificial Sequence
  gRNA target
tttgcccccagcgcttcagcgttc

SEQ ID NO: 25 - DNA, 24 bp, Artificial Sequence
  gRNA target
aaacgggggtcgcgaagtcgcaag

SEQ ID NO: 26 - DNA, 73 bp, SARS-CoV-2
ggggaacttctcctgctagaatgggctttgctgctgcttgacagattgaaccagcttgagagcaaaatgtctg

SEQ ID NO: 27 - DNA, 73 bp, SARS-CoV
ggggaaattctcctgctcgaatgggcgctattgctgctagacagattgaaccagcttgagagcaaagtttctg

SEQ ID NO: 28 - DNA, 80 bp, MERS-CoV
caggaaattctacccgcggcacttctccagggatctactttaccttgatcttctgaacagactacaagcccttgagtctg

SEQ ID NO: 29 - DNA, 76 bp, Human-CoV-0C43
gtagagccaattctggcaatagaacccctaccaaattgctagtcttgttctggcaaaacttggcaaggatgccact

SEQ ID NO: 30 - DNA, 73 bp, Human-CoV-HKU1
gtagaagtaattctaattttagacattcagagagatcgctaatcttgttttagccaagcttggtaaagattct

SEQ ID NO: 31 - DNA, 75 bp, Human-coV-229E
ctcggaatccttcaagtgacagaaaccatagttgctgcggctcttaaatctttaggttttgacaagcctcaggaa

SEQ ID NO: 32 - DNA, 74 bp, Human-CoV-NL63
ctcgtagcacttcaagacaacagtctcgcacgatcttgttgctgctgttactttggctttaaagaacttaggtt

SEQ ID NO: 33 - DNA, 80 bp, SARS-CoV-2
ccctgtgggttttacacttaaaaacacagtctgtaccgtctgcggtatgtggaaaggtcagtcagctgatgcacaatcgt

SEQ ID NO: 34 - DNA, 80 bp, SARS-CoV
cccagtgggttttacacttagaaacacagtctgtaccgtctgcggaatgtggaaagggcagtctgcggatgcatcaacgt

SEQ ID NO: 35 - DNA, 80 bp, MERS-CoV
ccctgtgggattttgtttgtcaaataccccctgtaatgtctgtcaatattggattgggccccaatctaaagattccaatt

SEQ ID NO: 36 - DNA, 79 bp, Human-Cov-0C43
tcctgtgtcttatgttttgacacatgatgtttgtcgagtttgtggattttggcgggagttcaatcaaaagatactaatt

SEQ ID NO: 37 - DNA, 79 bp, Human-CoV-HKU1
tcctattctttatgtgttaacacatgatgtttgtcaagtctgtggtttttggagagagttcaatctaaagatttaaatt

SEQ ID NO: 38 - DNA, 68 bp, Human-CoV-229E
tcctgattgtgttgattgccatgacgagatgtgtattttgcattgtattccaaacacggcttttggac

SEQ ID NO: 39 - DNA, 80 bp, Human-CoV-NL63
ttcttcaactcgtaacaactcacgagactcttctcgtagcacttcaagacaacagtccttcagatcttgttgctgctgtt

(19) United States
(12) Patent Application Publication
(10) Pub. No.: US 2023/0183817 A1
(43) Pub. Date: Jun. 15, 2023

(54) CRISPR-BASED ASSAY FOR DETECTING PATHOGENS IN SAMPLES

(71) Applicant: The Administrators of the Tulane Educational Fund, New Orleans, LA (US)

(72) Inventors: Tony Ye HU, New Orleans, LA (US); Bo NING, New Orleans, LA (US); Zhen HUANG, New Orleans, LA (US)

(21) Appl. No.: 17/926,129

(22) PCT Filed: May 20, 2021
(86) PCT No.: PCT/US21/33497

§ 371 Date: Nov. 17, 2022

Related U.S. Application Data
(60) Provisional application No. 63/027,530, filed on May 20, 2020.

(51) Int. Cl. C12Q 1/6888; C12Q 1/70; C12Q 1/6806; C12Q 1/6816; C12N 15/11

(52) U.S. Cl. CPC C12Q 1/6888; C12Q 1/701; C12Q 1/6806; C12Q 1/6816; C12N 15/11; C12N 2310/20; C12Q 2600/16

(57) ABSTRACT

The present disclosure describes a method for detecting the presence of pathogens, including SARS-CoV-2, in a sample. The method utilizes CRISPR effector proteins along with a guide RNA and a reporter molecule. RNAs in the sample are first optionally extracted and reverse transcribed, followed by amplification, such that when the guide RNA hybridizes with a target nucleotide fragment in the amplified DNA, the CRISPR effector protein cleaves the reporter molecule, resulting in a detectable signal.

This application claims priority to U.S. App. No. 63/027,530, filed May 20, 2020, which is incorporated herein in its entirety for all purposes.

### FEDERALLY SPONSORED RESEARCH STATEMENT

Not applicable.

### FIELD OF THE DISCLOSURE

The disclosure generally relates to a method of detecting SARS-CoV-2 (pathogen of CoVID-19) in a sample, and more particularly relates to a method of detecting SARS-CoV-2 in a sample using CRISPR-based reporter system, and a one-step detection method and system where results can be observed on a mobile phone.

### BACKGROUND OF THE DISCLOSURE

SARS-CoV-2, first detected in China's Hubei province late in 2019, rapidly spread from its initial outbreak site to produce a pandemic (Zhou et al. 2020), and has to date been detected in more than 200 countries, where it has infected more than 2 million confirmed people worldwide with more than 188,000 death so far.

However, disease control efforts are hindered by multiple factors, including difficulty in rapidly producing the number diagnostic tests required for such efforts, the apparent limited diagnostic sensitivity of current tests, and the technical expertise required to obtain valid results with them. Large scale testing appears to be essential since estimates indicate that large numbers of mild, asymptomatic or pre-symptomatic COVID-19 cases are not detected by current testing efforts. In the U.S., one estimate suggests that only 1.6% of COVID-19 cases have been detected, while another study estimates that about 17.9% of individuals infected with SARS-CoV-2 are asymptomatic at their first positive test and may not develop symptoms for up to two weeks. These are alarming statistics, since one estimate suggests that an infected individual may infect 5.6 additional people on average, and that individuals with asymptomatic or pre-symptomatic SARS-CoV-2 infections may be as infectious as those with symptomatic cases.

Therefore, ultrasensitive, inexpensive and high-throughput testing methods are thus required to allow large-scale screening efforts for case identification, isolation, and potential contact tracing of individuals, in order to improve local containment and to inform regional disease control efforts. Due to limited testing capacity, most countries and regions have prioritized testing for symptomatic and at-risk individuals. However, most symptoms associated with COVID-19, such as fever and cough, are non-specific and do not distinguish COVID-19 cases from individuals with other respiratory infections.

Nucleic acid tests employing reverse transcriptase polymerase chain reaction (RT-PCR) are the primary means used to diagnose COVID-19 using respiratory samples. Reverse transcription and PCR amplification can be consolidated into a single reaction to allow one-step assays that provide rapid, reproducible and high-throughput results, and this approach is used by the US CDC in its one-step real time RT-PCR SARS-CoV-2 assay. However, real-time quantitative PCR assays require well-trained personnel and expensive laboratory instruments to obtain accurate and robust results, which would limit their practical application outside well-equipped facilities.

As such, there is the need for rapid and ultrasensitive CoVID-19 diagnostic assay that are capable of high-throughput analyses without high degree of technical expertise or sophisticated equipment.

Additionally, currently collecting samples from a subject by either nasopharyngeal swab or nasal swab still poses transmission risk to medical personnel. When processing the samples, the step of extracting RNAs also requires proper training and handling of the samples that may not be practical for point-of-care operations.

Therefore, there is the need for a highly sensitive test for SARS-CoV-2 that has high specificity and quick turn-around rate, compatible with saliva samples, and without the RNA-extracting step.

### SUMMARY OF THE DISCLOSURE

In one embodiment, a method for detecting the presence of SARS-CoV-2 in a sample is described. The method comprises the steps of: optionally extracting RNAs from a sample; reverse-transcribing the RNAs into a mixture of DNAs; amplifying a SARS-CoV-2-specific target nucleic acid sequence from the mixture of DNAs; and detecting presence of the SARS-CoV-2-specific target nucleic acid sequence using a CRISPR-mediated system; wherein said CRISPR-mediated system comprises a CRISPR effector protein, a guide RNA (gRNA) that hybridizes with the SARS-CoV-2-specific target nucleic acid fragment, and a reporter molecule that is detectable on cleavage by said CRISPR effector protein.

In one embodiment, the amplification and detecting steps are combined in one single step. By using a mixture of amplification ingredients and CRISPR ingredients, these two steps may be performed at once instead of two separate steps.

In another aspect of this disclosure, a method of detecting the presence of SARS-CoV-2 in a sample is described. The method comprising the steps of: extracting RNAs from a sample; reverse-transcribing said RNAs into a DNA mixture and amplifying a SARS-CoV-2 target DNA sequence from said DNA mixture, wherein a pair of primers are used, and wherein the primers are SEQ ID NOs. 1&2, 4&5, 11&12, 13&14, 15&16, 17&18, 19&20 or 21&22; and detecting presence of the SARS-CoV-2 target DNA fragment in said amplified DNA using a CRISPR-mediated system; wherein said CRISPR-mediated system comprises Cas12a, a guide RNA (gRNA), and a reporter molecule that is detectable on cleavage by Cas12a.

In one embodiment, the extracting step can further comprise depleting human RNA using anti-human RNA antibodies. In one embodiment, the extracting step can further comprise enriching SARS-CoV-2 RNAs by using anti-SARS-CoV-2 RNA antibodies.

In one embodiment, the amplifying step is carried out using polymerase chain reaction (PCR), recombinase polymerase amplification (RPA), nucleic acid sequence-based amplification (NASBA), rolling circle amplification (RCA), or loop-mediated isothermal amplification (LAMP). In one embodiment, the amplifying step is carried out using PCR.

In one embodiment, CRISPR effector protein is selected from a group consisting of Cas12a, Cas9 and Cas13. In one embodiment, the CRISPR effector protein is Cas12a.

In one embodiment, the reporter molecule is a single-stranded DNA or a single-stranded RNA labeled with fluorescence and quencher, gold nanoparticles, or biotin-FAM. In one embodiment, the reporter molecule is 5-6-FAM-TTTTTTTTTTTT-BHQ1 (SEQ ID NO. 7).

In one embodiment, the sample is obtained from nasopharyngeal swap, oropharyngeal swab, nasopharyngeal wash, nasopharyngeal aspirate, nasal aspirate, nasal mid-turbinate swab, bronchoalveolar lavage, tracheal aspirate, pleural fluid, lung biopsy, sputum, or saliva.

In one embodiment, the target DNA sequence is a portion of N gene, E gene, M gene, S gene or ORFlab gene from the SARS-CoV-2 genome.

In one embodiment, more than one target DNA sequences are amplified by using different primer pairs.

In one embodiment, the primer pairs used in the amplification step is SEQ ID NOs. 1&2, 3&4, 5&6, 7&8 and 9&10, 11&12 and 13&14.

In one embodiment, the gRNA has at least one of the following sequences: SEQ ID NOs. 15, 16 and 17.

In another aspect of this disclosure, a method of detecting SARS-CoV-2 in a sample is described. The method comprises: adding the sample to a lysis solution to yield a lysate solution; mixing the lysate solution with a reaction solution, wherein the reaction solution comprises reagents for reverse-transcribing RNAs in the lysate solution into DNAs, and reagents for detecting a target DNA; and detecting presence of the SARS-CoV-2-specific target nucleic acid sequence.

In one embodiment, the detecting step is carried by using a smartphone. A smartphone-based fluorescent assay reader is also described, in which a smartphone camera can be used to capture fluorescent images of the samples after being excited by an incident radiation.

The present disclosure describes a CRISPR-based assay that utilizes a custom CRISPR Cas12a/gRNA complex and a fluorescent probe to amplify target amplicons produced by standard RT-PCR or isothermal recombinase polymerase amplification (RPA), to allow sensitive detection at sites not equipped with real-time PCR systems required for qPCR diagnostics. This approach allows sensitive and robust detection of SARS-CoV-2 positive samples, with a sample-to-answer time of less than one hour, and a limit of detection of 2 copies per sample.

Additionally, by optimizing the lysis, amplification and detection conditions, the detection process can be carried out using saliva samples at or near room temperature. The limit of detection is also comparable as compared to

Rapid and ultrasensitive COVID-19 diagnostic assays that are capable of high-throughput analyses and do not require a high degree of technical expertise or sophisticated equipment are required to expand COVID-19 testing capacity. CRISPR-Cas/gRNA complexes have recently been used to sensitively detect nucleic acids, including in those derived from human pathogens.

CRISPR (Clustered Regularly Interspaced Short Palindromic Repeats) is a family of DNA sequences found within the genomes of prokaryotic organisms such as bacteria and archaea. These sequences are derived from DNA fragments of bacteriophages that have previously infected the prokaryote and are used to detect and destroy DNA from similar phages during subsequent infections, and therefore they serve as an important part of the prokaryotes' immune system.

CRISPR-associated protein 9 (“Cas9”) is an enzyme that uses CRISPR sequences as a guide to recognize and cleave specific strands of DNA that are complementary to the CRISPR sequence. The Cas9 endonuclease is a four-component system that includes two small crRNA molecules and trans-activating CRISPR RNA (tracrRNA). The two RNA sequences were fused into a single guide RNA (gRNA), which guides Cas9 to find and cut the DNA target specified by the guide RNA. Cas9 enzymes together with CRISPR sequences form the basis of a technology known as CRISPR-Cas9 that can be used to edit genes within organisms. By manipulating the nucleotide sequence of the guide RNA, the artificial Cas9 system could be programmed to target any DNA sequence for cleavage.

Nuclease Cas12a (formerly known as Cpf1), another member of the Cas family, showed several key differences from Cas9 including: causing a ‘staggered’ cut in double stranded DNA as opposed to the ‘blunt’ cut produced by Cas9, relying on a ‘T rich’ protospacer adjacent motifs, thus providing alternative targeting sites to Cas9, and requiring only one CRISPR RNA for successful targeting.

Cas13 (including four subtypes: Cas13a-d) functions similarly to Cas9, using a ˜64-nt guide RNA to encode target specificity. The Cas13 protein complexes with the guide RNA via recognition of a short hairpin in the crRNA, and target specificity is encoded by a 28 to 30-nt spacer that is complementary to the target region. In addition to programmable RNase activity, all Cas13s exhibit collateral activity after recognition and cleavage of a target transcript, leading to non-specific degradation of any nearby transcripts regardless of complementarity to the spacer.

In one embodiment, the CRISPR effector protein is Cas12a (formerly known as Cpf1), Cas9 or Cas13. However, other CRISPR effector proteins can be used, as long as effective detection with high specificity can be achieved.

By properly designing the gRNA by varying its length and location in a particular gene, one can thus target a DNA fragment with the desirable specificity and sensitivity. Therefore, with the CRISPR-Cas-gRNA method of this disclosure, highly sensitive and specific detection of SARS-CoV-2 in a sample can be achieved in a matter of hours. It is expected to be able to detect as low as two copies of SARS-CoV-2 RNA in a sample.

The method can also be used to distinguish different strains of SARS-CoV-2 by targeting strain-specific RNA fragments. It has been reported that more than six different strains of SARS-CoV-2 exist around the globe. Although it is still unclear if a different strain would cause clinical difference, it may be beneficial to identify different strains of SARS-CoV-2 based on their genetic variance. Supportive CT scan and clinical findings can also be used to establish the diagnosis.

Further variation can be carried out in order to enhance SARS-CoV-2 detection. In one embodiment, the RNA extracted from the sample can be further enriched by first treated with anti-RNA antibodies. In another embodiment, the RNA extracted from the sample can be further treated with anti-human RNA antibodies in order to deplete human RNA from the sample.

In one embodiment, anti-human RNA antibody is anti-(U1) small nuclear RNA) antibody. However, other antibodies or proteins can be used, as long as it can bind to human-specific RNAs.

In another aspect of this disclosure, a method of detecting the presence of a pathogen in a sample is described. The method comprises the steps of: extracting DNAs or RNAs from a sample; optionally reverse-transcribing said RNAs into a DNA mixture, amplifying a target DNA sequence from said DNA mixture or from the extracted DNAs in step a), wherein a pair of primers matching a portion of said target DNA are used; and detecting presence of the target DNA fragment in said amplified DNA using a CRISPR-mediated system; wherein said CRISPR-mediated system comprises Cas12a, a guide RNA (gRNA), and a reporter molecule that is detectable on cleavage by Cas12a, and wherein said gRNA matches a portion of said target DNA; and wherein said pathogen is selected from a group consisting of: human coronavirus 229E, human coronavirus OC43, human coronavirus HKU1, human corona virus NL63, SARS-coronavirus, MERS-coronavirus, Adenovirus (e.g. C1 Ad. 71), Human Metapneumovirus (hMPV), Parainfluenza virus 1-4, Influenza A & B, Enterovirus (e.g. EV68), Respiratory syncytial virus, Rhinovirus, Chlamydia pneumoniae, Haemophilus influenzae, Legionella pneumophila, Mycobacterium tuberculosis, Streptococcus pneumoniae, Streptococcus pyogenes, Bordetella pertussis, Mycoplasma pneumoniae, Pneumocystis jirovecii (PJP), Candida albicans, Pseudomonas aeruginosa, Staphylococcus epidermis, Staphylococcus salivarius.

In another aspect of this disclosure, a fluorescent assay device is described. The fluorescent assay device comprises: a phone holder; a lens; a fluorescence filter; an adaptor to receive a reaction chip; a light source, wherein the light source emits a light to the reaction chip to excite fluorescence signals; and a temperature control module capable of controlling temperatures on the reaction chip; wherein the phone holder receives a smartphone having a camera, and the lens and the fluorescence filter are aligned between the camera and the reaction chip.

In one embodiment, the fluorescent assay device may further comprise a controller, wherein the controller controls the light source and the temperature control module.

In one embodiment, the reaction chip comprises a plurality of wells for receiving reagents and samples. In one embodiment, the plurality of wells are connected through microfluidic channels that may be controlled by microfluidic valves. In one embodiment, the controller controls the fluid flow within the microfluidic channels between the plurality of wells.

In one embodiment, the fluorescent assay device may further comprise a communication module operatively connected to the controller, wherein the communication module establishes a communication with a smartphone, wherein the communication is wired or wireless.

In one embodiment, the smartphone controls the light source, the temperature control module through the communication with the controller, and the camera.

In one embodiment, the smartphone analyzes images captured by the camera. In one embodiment, the smartphone is capable of transmitting the analyzed results to a remote server or a remote user.

In one embodiment, the phone holder is adjustable in size to accommodate different phones.

In one embodiment, the fluorescent assay device further comprises a batter power source.

Various RNA and DNA amplification techniques can be used, as each technique has its advantages and disadvantages. The method of this disclosure can employ the common reverse transcription to convert RNA into DNA, followed by DNA amplification techniques, such as PCR, RPA, RCA, LAMP, etc., as well as any newly developed amplification methods.

The methods combine reverse transcription, DNA amplification and CRISPR detection. In amplification steps, hybridization enhancer component in reaction buffer can be used to enhance specific primer-template hybridization during every cycle of DNA amplification, preventing mispriming and improving DNA amplification specificity and yield. We expect to be able to amplify SARS-CoV-2 RNA from a single copy in a sample for CRISPR detection.

Hybridization enhancers can be further carried over to the CRISPR detection step, as they can further improve hybridization between of guide RNA and target sequences and reduce mismatch. Such enhancers are expected to be able to stabilize and enhance activities of CRISPR proteins and amplify signal.

In one embodiment, the hybridization enhancers are the thermostable AccuPrime accessory proteins. These enhance specific primer-template hybridization during every cycle of PCR, preventing mispriming and improving PCR specificity and yield. Other hybridization enhancers can also be used, as long as they can enhance specificity of hybridization. Non-limiting examples of hybridization enhancers include anionic polymers, in situ hybridization buffers and similar buffer components, AccuPrime accessory proteins, ULTRAhyb™ Ultrasensitive Hybridization Buffer and the like.

As used herein, “CRISPR proteins” or “CRISPR effector protein” or “CRISPR enzymes” refers to Class 2 CRISPR effector proteins including but not limited to Cas9, Cas12a (formerly known as Cpf1), Csn2, Cas4, C2c1, Cc3, Cas13a, Cas13b, Cas13c, Cas13d. In one embodiment, the CRISPR effector proteins described herein are preferably Cpf1 effector proteins.

As used herein, “guide RNA” or “gRNA” refers to the non-coding RNA sequence that binds to the complementary target DNA sequence to guide the CRISPR-Cas system in close contact with the target DNA strand.

As used herein, a “reporter molecule” refers to a single-stranded DNA or single-stranded RNA that is labeled with fluorescence and quencher, gold nanoparticles or biotin-FAM, and the dissociation of the reporter can be detected by either a fluorescence reader or colorimetric change in e.g., a paper lateral flow assay or spectrometer, and the like.

As used herein, the “target fragment” is a portion of a SARS-CoV-2-specific RNA sequence that has been reverse-transcribed into a cDNA sequence. For example, portions from N gene or ORFlab gene of SARS-CoV-2 can be used as the target fragment.

As used herein, “reverse transcription” refers to converting an RNA sequence back to a cDNA sequence using reverse transcriptase.

As used herein, “DNA amplification” or “nucleic acid amplification” refers to natural and artificial processes by which the number of copies of a gene or a fragment of DNA is increased without a proportional increase in other genes.

As used herein, “polymerase chain reaction” or “PCR” refers to a method of amplifying a specific target region of a DNA strand by using a DNA polymerase and two primers (forward and reverse) that are complimentary to each end of the target region, along with dNTPs.

As used herein, “recombinase polymerase amplification” or RPA refers to a method of amplifying a specific target region using a recombinase, a single-stranded DNA-binding protein and strand-displacing polymerase. The recombinase pairs oligonucleotide primers with homologous sequence in duplex DNA, and the single-stranded DNA-binding protein binds to replaced strands of DNA to prevent the primers from being displaced. An optimal temperature at 32-45° C., the reaction progresses rapidly and results in specific DNA amplification without the need for thermal or chemical melting required by PCR.

As used herein, “nucleic acid sequence-based amplification” or NASBA refers to a primer-dependent method for continuously amplifying nucleic acids, particularly RNA sequences, in a single mixture at one temperature. Three enzymes are used: a reverse transcriptase, a RNase H, and T7 RNA polymerase. Two primers are used: the first primer includes a 3′-terminal sequence that is complementary to a target sequence and a 5′-terminal sense sequence of a promoter that is recognized by the T7 RNA polymerase; and the second primer includes a sequence complementary to the P1-primed DNA strand. First, an RNA template is given to the reaction mixture, where the first primer attaches to its complementary site at the 3′ end of the template. The reverse transcriptase synthesizes the opposite, complementary DNA strand, extending the 3′ end of the primer, moving upstream along the RNA template. At this time, RNAse H destroys the RNA template from the DNA-RNA compound (RNAse H only destroys RNA in RNA-DNA hybrids, but not single-stranded RNA). The second primer then attaches to the 5′ end of the (antisense) DNA strand. Afterwards, the reverse transcriptase again synthesizes another DNA strand from the attached primer resulting in double stranded DNA, when the T7 RNA polymerase binds to the promoter region on the double stranded DNA. Since T7 RNA polymerase can only transcribe in the 3′ to 5′ direction, the sense DNA is transcribed, and an anti-sense RNA is produced. This is repeated, and the polymerase continuously produces complementary RNA strands of the template which results in amplification.

Now a cyclic phase can begin similar to the previous steps. Here, however, the second primer first binds to the (−)RNA, and the reverse transcriptase now produces a (+)cDNA/(−)RNA duplex. RNAse H again degrades the RNA and the first primer binds to the now single stranded +(cDNA), followed by the reverse transcriptase producing the complementary (−)DNA and creating a dsDNA duplex. Lastly, the T7 polymerase binds to the promoter region, produces (−)RNA, and the cycle is complete.

As used herein, “rolling circle amplification” or RCA refers to an isothermal enzymatic process where a short DNA or RNA primer is amplified to form a long single stranded DNA or RNA using a circular DNA template and special DNA or RNA polymerases. The RCA product is a concatemer containing tens to hundreds of tandem repeats that are complementary to the circular template.

As used herein, “loop-mediated isothermal amplification” or LAMP refers to a single tube DNA amplification method, where the target sequence is amplified at a constant temperature of 60-65° C. using either two or three sets of primers and a polymerase with high strand displacement activity in addition to a replication activity. Typically, 4 different primers are used to amplify 6 distinct regions on the target gene, which increases specificity. An additional pair of “loop primers” can further accelerate the reaction.

As used here, “reporter molecule” refers to a molecule having nucleotides linked to a detectable reporter group, such that when the nucleotides hybridize with a matching sequence, the reporter group produces detectable signals. Non-limiting reporter molecule includes a single-stranded DNA or RNA labeled with fluorescence and quencher, gold nanoparticles, biotin-FAM.

As used herein, “anti-human DNA antibodies” refers to anti-nuclear antibodies that target double stranded human DNA as antigen.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| CRISPR | Clustered regularly interspaced short |
| | palindromic repeats |
| gRNA | Guide RNA |
| LAMP | Loop-mediated isothermal amplification |
| NASBA | Nucleic acid sequence-based amplification |
| HCoV-229E | Human coronavirus 229E |
| HCoV-OC43 | Human coronavirus OC43 |
| HCoV-HKU1 | Human coronavirus HKU1 |
| HCoV-NL63 | Human coronavirus NL63 |
| SARS | Severe acute respiratory syndrome |
| MERS | Middle East respiratory syndrome |
| PAM | Protospacer adjacent motif |
| PCR | Polymerase chain reaction |
| RCA | Rolling circle amplification |
| RPA | Recombinase polymerase amplification |

### BRIEF DESCRIPTION OF THE DRAWINGS

FIG. 1A. Schematic illustration of a CRISPR-FDS assay for detection of SARS-CoV-2 RNA in clinical samples according to one embodiment of this disclosure.

FIG. 1B. SARS-CoV-2 genome map of COVID-19 CRISPR-FDS target sequences.

FIG. 1C. Sites in ORF1ab gene and the N protein gene that are detected COVID-19 CRISPR-FDS. following

FIG. 1D-F. Normalized CRISPR-FDS photoluminescent (PL) signal from SARS-CoV-2 RNA positive (109 copies/sample) and negative control (polyA carrier RNA) samples following target amplification by RT-PCR or RPA, by RT-PCR for each assay target, and by RT-PCR for related beta coronavirus species (109 copies/sample). Bar graph data represents the mean±SD, of three experimental replicates.

FIG. 2A-C. CRISPR-FDS signals that are (A) Substrate-dependent, (B) temperature-dependent, and (C) target-dependent. An aliquot containing 109 target amplicon copies, or an equivalent amount of poly A carrier RNA were analyzed as positive (+) and negative (−) control samples, respectively. Data presented in the top rows of each panel and the bottom row of (C) are normalized to the highest signal intensity detected in the corresponding experiment. Bar graph data represents the mean ±SD, of three experimental replicates. (ns, P>0.05; ****, P<0.0001)

FIG. 3A-C. COVID-19 CRISPR-FDS analytical and diagnostic performance. Limit of detection (LOD) samples containing the indicated number of viral genomes after amplification by (A) RT-PCR and (B) RT-RPA for COVID-19 CRISPR-FDS analysis or by (C) qPCR, indicated significant differences and undetermined (UD) results.

FIG. 3D. RT-PCR COVID-19 CRISPR-FDS results for a cohort of 28 individuals with suspected COVID-19 cases, run in parallel with blank (BC; nuclease free water), negative (NC; carrier RNA) and positive (PC; 109 target amplicon copies) control samples, where the dashed line indicates the threshold for a positive result. Results depict the mean ±SD of three experimental replicates.

FIG. 3E. Comparison of SARS-CoV-2 test results for matching patient samples analyzed by CRISPR-FDS, or by a state (qPCR 1) and a clinical testing laboratory (qPCR 2). (ns, P>0.05; *, P<0.05; **, P<0.01; ***, P<0.001; ****, P<0.0001).

FIG. 4. Schematic illustration of a CRISPR-FDS assay for detection of SARS-CoV-2 RNA in clinical samples according to another embodiment of this disclosure.

FIG. 5A. Schematic illustration of a 3D-printed smartphone fluorescence reader.

FIG. 5B. Workflow of a saliva-based on-chip CRISPR-FDS smartphone assay.

FIG. 5C. An example of CRISPR-FDS assay fluorescent signal image captured with a 525 nm filter using a cellphone.

FIG. 5D. Standard curve of the on-chip CRISPR-FDS saliva test read by the smartphone device.

FIG. 5E. Comparison SARS-CoV-2 viral load in saliva samples read by the smartphone device and by RT-PCR.

FIG. 5F. Correlation of smartphone CRISPR-FDS and RT-qPCR assay results for saliva samples from 103 COVID-19 cases, indicating the linear regression line (solid) and the limits of its 95% confidence interval (dashed line). Data represent the mean±SD of three replicates.

### DETAILED DESCRIPTION

The disclosure provides novel method of detecting the presence of a pathogen in a sample by first amplifying a target DNA sequence followed by detection by a CRISPR-mediated system. In the case of a pathogen having a RNA genome, a reverse-transcription step is further performed. The pathogens that can be detected by this disclosure include human coronavirus 229E, human coronavirus OC43, human coronavirus HKU1, human corona virus NL63, SARS-coronavirus, MERS-coronavirus, adenovirus, Human Metapneumovirus (hMPV), Parainfluenza virus 1-4, Influenza A & B, Enterovirus (e.g. EV68), Respiratory syncytial virus, Rhinovirus, Chlamydia pneumoniae, Haemophilus influenzae, Legionella pneumophila, Mycobacterium tuberculosis, Streptococcus pneumoniae, Streptococcus pyogenes, Bordetella pertussis, Mycoplasma pneumoniae, Pneumocystis jirovecii (PJP), Candida albicans, Pseudomonas aeruginosa, Staphylococcus epidermis, Staphylococcus salivarius.

Human coronavirus 229E is a species of coronavirus which infects humans and bats. It is an enveloped, positive-sense, single-stranded RNA virus which enters its host cell by binding to the APN receptor. Various sequences of Human coronavirus 229E are available on GenBank, for example at Accession No. KF514433.1 for strain 229E/human/USA/933-40/1993 complete genome.

Human coronavirus OC43 is a member of the species Betacoronavirus 1 which infects humans and cattle. The infecting coronavirus is an enveloped, positive-sense, single-stranded RNA virus which enters its host cell by binding to the N-acetyl-9-O-acetylneuraminic acid receptor. Various sequences of Human coronavirus OC43 are available on GenBank, for example at Accession No. KX344031.1 for isolate LRTI_238 complete genome.

Human coronavirus HKU1 is a species of coronavirus which originated from infected mice. In humans, infection results in an upper respiratory disease with symptoms of the common cold, but can advance to pneumonia and bronchiolitis. The virus is an enveloped, positive-sense, single-stranded RNA virus which enters its host cell by binding to the N-acetyl-9-O-acetylneuraminic acid receptor. It has the Hemagglutinin esterase (HE) gene, which distinguishes it as a member of the genus Betacoronavirus and subgenus Embecovirus. Various sequences of Human coronavirus HKU1 are available on GenBank, for example at Accession No. KF430201.1 for strain HKU1/human/USA/HKU1-18/2010 complete genome.

Human coronavirus NL63 is a species of coronavirus. It is an enveloped, positive-sense, single-stranded RNA virus which enters its host cell by the ACE2 receptor. Infection with the virus has been confirmed worldwide, and has an association with many common symptoms and diseases. Associated diseases include mild to moderate upper respiratory tract infections, severe lower respiratory tract infection, croup and bronchiolitis. Various sequences of Human coronavirus NL63 are available on GenBank, for example at Accession No. KF530114.1 for strain NL63/human/USA/891-4/1989 complete genome.

Severe acute respiratory syndrome coronavirus coronavirus (SARS-CoV), is a strain of virus that causes severe acute respiratory syndrome (SARS). It is an enveloped, positive-sense, single-stranded RNA virus which infects the epithelial cells within the lungs. The virus enters the host cell by binding to the ACE2 receptor. It infects humans, bats, and palm civets. Various sequences of SARS-CoV are available on GenBank, for example at Accession No. DQ898174.1 for strain CV7 complete genome.

Middle East respiratory syndrome coronavirus (MERS-CoV) is a species of coronavirus which infects humans, bats, and camels. The infecting virus is an enveloped, positive-sense, single-stranded RNA virus which enters its host cell by binding to the DPP4 receptor. Various sequences of MERS-CoV are available on GenBank, for example at Accession No. MH734115.1 for isolate camel/Kenya/C1272/2018 complete genome.

Adenoviruses are a group of medium-sized (90-100 nm), nonenveloped viruses with an icosahedral nucleocapsid containing a double stranded DNA genome. Adenoviruses are common viruses where 57 accepted human adenovirus types (HAdV-1 to 57) in 7 species (Human Adenovirus A to G) have been identified. The genome sequences of human adenovirus C1 (hAdV-C1) is available on GenBank, for example at Accession Nos. MF177731 or MF1777732, and primers for amplification can be designed accordingly.

Human Metapneumovirus (hMPV) infects airway epithelial cells in the nose and lung. hMPV is a negative-sense single-stranded RNA virus of the family Pneumoviridae and is closely related to the Avian metapneumovirus subgroup C. The genomic organization of hMPV lacks the non-structural genes, NS1 and NS2, and the hMPV antisense RNA genome contains eight open reading frames in slightly different gene order than RSV (viz. 3′-N-P-M-F-M2-SH-G-L-5′). hMPV genome is available on GenBank, for example at Accession No. NC_039199, and primers can be designed accordingly to amplify a portion thereof.

Human parainfluenza viruses (HPIVs) are the viruses that cause human parainfluenza. HPIVs are a paraphyletic group of four distinct single-stranded RNA viruses belonging to the Paramyxoviridae family. Virions are approximately 150-250 nm in size and contain negative sense RNA with a genome encompassing about 15,000 nucleotides. The genome of HPIVs has been fully sequenced and is available on GenBank, for example at Accession Nos. DI169299, DI169298, DI169297, etc.

Influenza, commonly known as “the flu”, is an infectious disease caused by an influenza virus. Influenza viruses are RNA viruses that make up four of the seven genera of the family Orthomyxoviridae: influenzavirus A-D, where subtypes A and B are most prevalent in human. The influenza A virus can be subdivided into different serotypes based on the antibody response to these viruses. The influenzavirus B mutates at a rate 2-3 times slower than type A and consequently is less genetically diverse, with only one influenza B serotype. Influenzavirus has been widely studied and sequenced. Depending on the subtype, various portions of the influenzavirus genome are available on GenBank, for example Accession Nos. EF100818 for subtype A polymerase PB1, DQ643813 for subtype A nonstructural protein 2, DQ643810 for subtype A neuraminidase, DQ643809 for subtype A hemagglutinin, D00004 for subtype B mRNA, etc.

Enterovirus is a genus of positive-sense single-stranded RNA viruses associated with several human and mammalian diseases. Serologic studies have distinguished 71 human enterovirus serotypes on the basis of antibody neutralization tests. Enterovirus is characterized by a single positive-strand genomic RNA. All enteroviruses contain a genome of approximately 7,500 bases and are known to have a high mutation rate due to low-fidelity replication and frequent recombination. Various sequences of enterovirus genomic RNA are available on GenBank, for example Accession No. NC_030454 for human enterovirus strain V13-0285.

Respiratory syncytial virus (RSV) causes infections of the lungs and respiratory tract. RSV is a medium-sized (120-200 nm) enveloped virus that contains a linear negative-sense RNA genome. RSV is divided into two antigenic subgroups, A and B, on the basis of the reactivity of the virus with monoclonal antibodies against the attachment (G) and fusion (F) glycoproteins. Subtype B is characterized as the asymptomatic strains of the virus that the majority of the population experiences. Genomic RNA sequences of RSV are available on GenBank, for example Accession No. NC_001803 for RSV genome.

Rhinovirus is the most common viral infectious agent in humans and is the predominant cause of the common cold. The three species of rhinovirus (A, B, and C) include around 160 recognized types of human rhinovirus that differ according to their surface proteins (serotypes). Rhinoviruses have single-stranded positive sense RNA genomes of between 7200 and 8500 nt in length. Human rhinoviruses are composed of a capsid that contains four viral proteins, VP1, VP2, VP3 and VP4. VP1, VP2, and VP3 form the major part of the protein capsid. The much smaller VP4 protein has a more extended structure, and lies at the interface between the capsid and the RNA genome. There are 60 copies of each of these proteins assembled as an icosahedron. Antibodies are a major defense against infection with the epitopes lying on the exterior regions of VP1-VP3. Various sequences of human rhinovirus are available on GenBank, for example Accession Nos. M121691 for serotype 1A protease, M12168 for serotype 14 LP protease, etc.

Chlamydia pneumoniae is a species of Chlamydia, an obligate intracellular bacterium that infects humans and is a major cause of pneumonia. Chlamydia pneumoniae is a small gram negative bacterium (0.2 to 1 m) that undergoes several transformations during its life cycle. C. pneumoniae has a complex life cycle and must infect another cell to reproduce; thus, it is classified as an obligate intracellular pathogen. Various sequences of C. pneumoniae are available on GenBank, for example

Haemophilus influenzae is a Gram-negative, coccobacillary, facultatively anaerobic pathogenic bacterium of the family Pasteurellaceae. H. influenzae is responsible for a wide range of localized and invasive infections. Clinical diagnosis of H. influenzae is typically performed by bacterial culture or latex particle agglutinations. Diagnosis is considered confirmed when the organism is isolated from a sterile body site. Various sequences of Haemophilus influenzae are available on GenBank, for example at Accession No. CP000672 for Haemophilus influenzae PittGG.

Legionella pneumophila is a thin, aerobic, pleomorphic, flagellated, non-spore-forming, Gram-negative bacterium of the genus Legionella. L. pneumophila is the causative agent of Legionnaires' disease. Sera have been used both for slide agglutination studies and for direct detection of bacteria in tissues using fluorescent-labelled antibody. Specific antibody in patients can be determined by the indirect fluorescent antibody test. ELISA and microagglutination tests have also used. Various sequences of Legionella pneumophila are available on GenBank, for example Accession No. RBGB01000022 for Legionella pneumophila strain NMB001853 501503-12_22 whole genome shotgun sequence.

Mycobacterium tuberculosis (M. tb) is a species of pathogenic bacteria in the family Mycobacteriaceae and the causative agent of tuberculosis. M. tuberculosis has a waxy coating on its cell surface primarily due to the presence of mycolic acid. M. tuberculosis has a remarkably slow growth rate, doubling roughly once per day. The most frequently used diagnostic methods for tuberculosis are the tuberculin skin test, acid-fast stain, culture, and polymerase chain reaction. Various sequences of Mycobacterium tuberculosis are available on GenBank, for example at Accession No. AP018036.1 for M. tuberculosis complete genome (strain HN-506).

Streptococcus pneumoniae is a Gram-positive, spherical bacteria, alpha-hemolytic (under aerobic conditions) or beta-hemolytic (under anaerobic conditions), facultative anaerobic member of the genus Streptococcus. S. pneumoniae is the main cause of community acquired pneumonia and meningitis in children and the elderly. Diagnosis is conventionally made based on clinical suspicion along with a positive culture from a sample from virtually any place in the body. In addition, molecular methods for the detection and identification of S. pneumoniae have been proposed. Various sequences of Streptococcus pneumoniae are available on GenBank, for example at Accession No. CP027540.1 for Streptococcus pneumoniae strain D39V chromosome, complete genome.

Streptococcus pyogenes is a species of Gram-positive, aerotolerant bacterium in the genus Streptococcus. These bacteria are extracellular, and made up of non-motile and non-sporing cocci. S. pyogenes causes an estimated 700 million GAS infections worldwide each year. While the overall mortality rate for these infections is 0.1%, over 650,000 of the cases are severe and invasive, and have a mortality rate of 25%. Early recognition and treatment are critical; diagnostic failure can result in sepsis and death. Various sequences of Streptococcus pyogenes are available on GenBank, for example at Accession No. AP014596.1 for Streptococcus pyogenes complete genome (strain M3-b).

Bordetella pertussis is a Gram-negative, aerobic, pathogenic, encapsulated coccobacillus of the genus Bordetella, and the causative agent of pertussis or whooping cough. Its virulence factors include pertussis toxin, adenylate cyclase toxin, filamentous haemagglutinin, pertactin, fimbria, and tracheal cytotoxin. Cell culture, ELISA and PCR are the current method of diagnosis. Various sequences of Bordetella pertussis are available on GenBank, for example at Accession No. CPO 11448.1 for strain B3921 complete genome.

Mycoplasma pneumoniae is a very small bacterium in the class Mollicutes. It is a human pathogen that causes the disease mycoplasma pneumonia, a form of atypical bacterial pneumonia related to cold agglutinin disease. M. pneumoniae is characterized by the absence of a peptidoglycan cell wall and resulting resistance to many antibacterial agents. The persistence of M. pneumoniae infections even after treatment is associated with its ability to mimic host cell surface composition. PCR is the most rapid and effective way to determine the presence of M. pneumoniae, however the procedure does not indicate the activity or viability of the cells present. Various sequences of Mycoplasma pneumoniae are available on GenBank, for example at Accession No. NZ_CP014267.1 for strain C267 chromosome, complete genome.

Pneumocystis jirovecii (previously P. carinii) is a yeast-like fungus of the genus Pneumocystis. The causative organism of Pneumocystis pneumonia, it is an important human pathogen, particularly among immunocompromised hosts. Various sequences of Pneumocystis jirovecii are available on GenBank, for example at Accession No. LFWA01000001.1 for RU7 supercont1.1, whole genome shotgun sequence.

Candida albicans is an opportunistic pathogenic yeast that is a common member of the human gut flora. It is one of the few species of the genus Candida that causes the human infection candidiasis, which results from an overgrowth of the fungus. C. albicans is the most common fungal species isolated from biofilms either formed on implanted medical devices or on human tissue. A mortality rate of 40% has been reported for patients with systemic candidiasis due to C. albicans. Various sequences of Candida albicans are available on GenBank, for example at Accession No. CM016738.1 for strain NCYC 4146 chromosome 1, whole genome shotgun sequence.

Pseudomonas aeruginosa is a common encapsulated, Gram-negative, rod-shaped bacterium that can cause disease in plants and animals, including humans. A species of considerable medical importance, P. aeruginosa is a multidrug resistant pathogen recognized for its ubiquity, its intrinsically advanced antibiotic resistance mechanisms, and its association with serious illnesses—hospital-acquired infections such as ventilator-associated pneumonia and various sepsis syndromes. Cell culturing is the primary method of detecting the presence of P. aeruginosa. Various sequences of Pseudomonas aeruginosa are available on GenBank, for example at Accession No. CP007224.1 for PA96 genome.

Staphylococcus epidermidis is a Gram-positive bacterium, and one of over 40 species belonging to the genus Staphylococcus. S. epidermidis is a particular concern for people with catheters or other surgical implants because it is known to form biofilms that grow on these devices. Cell culturing is the primary method of detecting the presence of S. epidermidis. Various sequences of Staphylococcus epidermidis are available on GenBank, for example at Accession No. NZ_JUKL00000000.1 for strain 997_SHAE, whole genome shotgun sequence.

Streptococcus salivarius is a species of spherical, gram-positive, facultative anaerobic bacteria that is both catalase and oxidase negative. S. salivarius colonizes the oral cavity and upper respiratory tract of humans just a few hours after birth, making further exposure to the bacteria harmless in most circumstances. However, S. salivarius in the bloodstream can cause sepsis in people with neutropenia. Various sequences of Streptococcus salivarius are available on GenBank, for example at Accession No. NZ_JWGR00000000.1 for strain 1003_SOLI, whole genome shotgun sequence.

The detection of these pathogens still heavily relies on cell culturing, which takes days to weeks to complete. Using the method of this disclosure, the CRISPR-mediated detection system can detect and identify the pathogens with high accuracy in a matter of hours. The primers used to reverse-transcribe and/or amplify can be specifically made based on the target sequence of the pathogens. A person skilled in the art can readily design the proper primers for reverse-transcribing RNA into DNA as well as amplifying the target DNA sequence.

In particular, this disclosure describes a method for detecting the presence of SARS-CoV-2 RNA in a sample. The method comprises the steps of: a) extracting RNA from a sample; b) reverse transcribing said RNAs into a DNA sequence; c) amplifying a target DNA sequence, and d) detecting presence of the target DNA sequence using a CRISPR-mediated system, wherein the CRISPR-mediated system comprises a CRISPR effector protein, a guide RNA that hybridizes with the target nucleic acid sequence, and a reporter molecule.

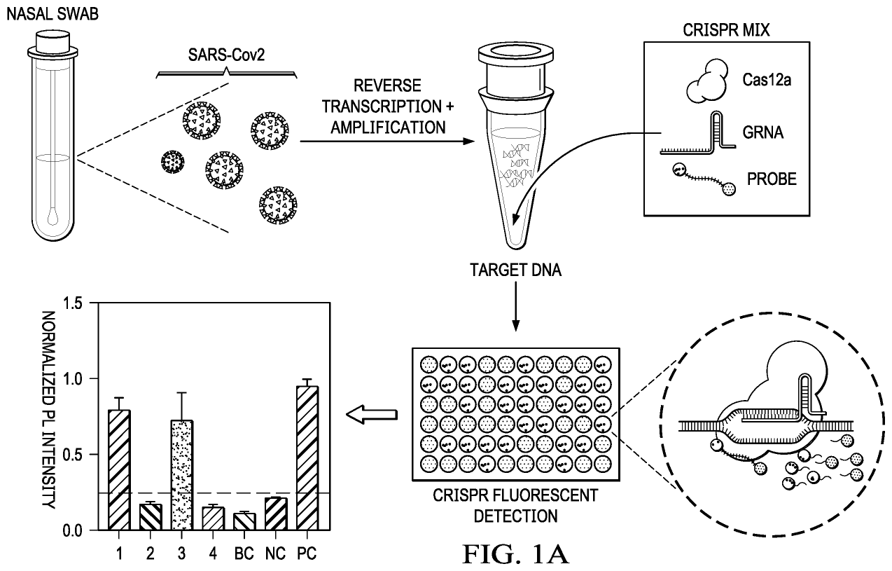

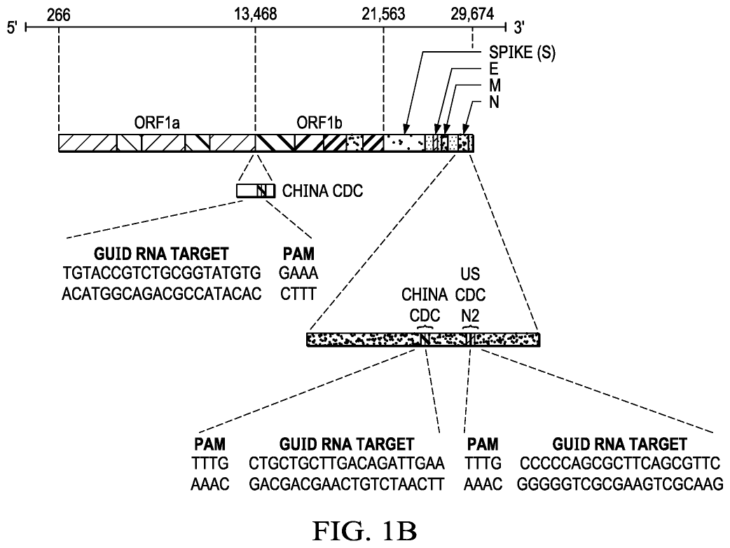

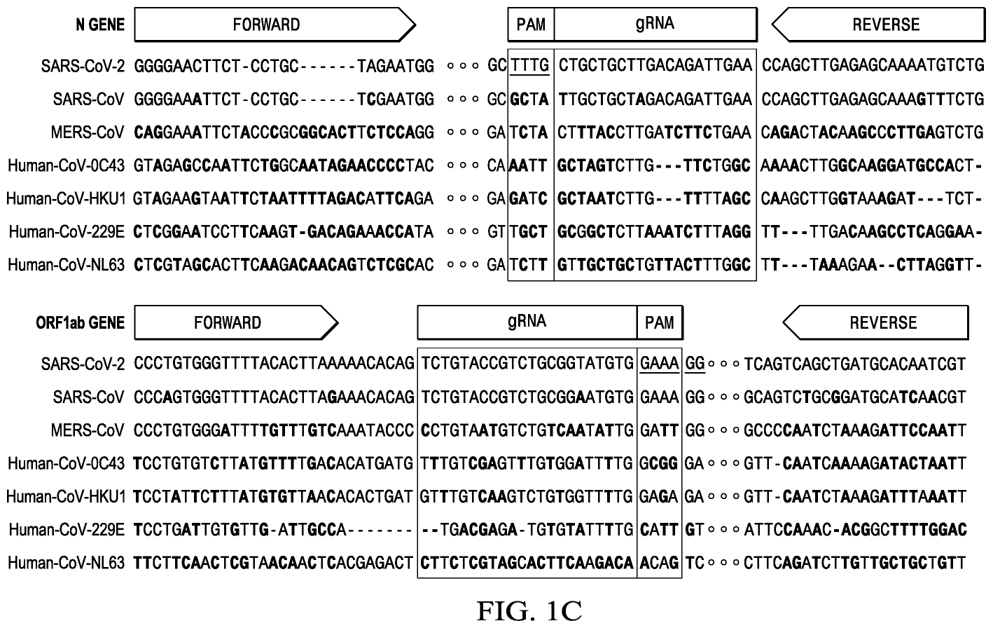

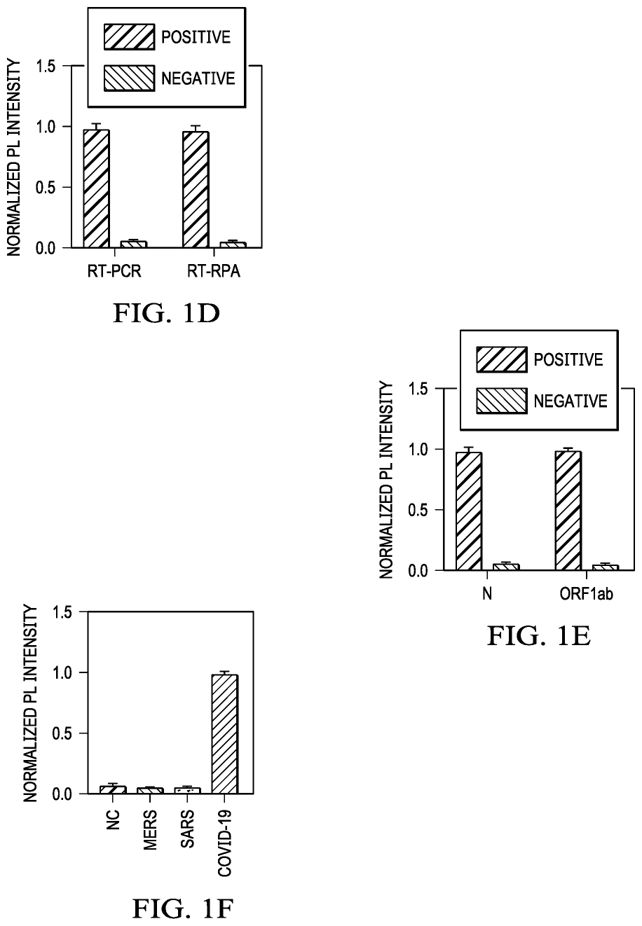

As shown in FIG. 1A, one-step reverse-transcriptase polymerase chain reaction (RT-PCR) or recombinase polymerase amplification (RT-RPA) methods are used to amplify viral cDNA target regions from RNA extracted from nasal swabs, and the resulting amplicons are transferred in their entirety to the gRNA/Cas12a-based CRISPR system for fluorescence detection. Recognition of the target amplicon by the gRNA/Cas12a complex, which is regulated by a target-specific synthetic gRNA, induces the gRNA/Cas12a complex to specifically cleaves the target amplicon and non-specifically cleave a reporter oligo modified with fluorescein and a quencher molecule at each terminus to produce a fluorescent signal. Notably, this method has a 1 hour sample to answer time, employs easily obtained reagents and equipment available in most clinical laboratories, can be readily automated to meet a demand for high-throughput testing, and has the potential for use in point-of-care settings if assay results are analyzed with a portable fluorescence reader.

Isothermal amplification methods (e.g., the recombinase polymerase amplification (RPA) and loop-mediated isothermal amplification (LAMP) methods, that can provide analytical sensitivities similar to PCR without a thermocycler requirement are being utilized in SARS-CoV-2 diagnostics currently under development. One recently published report, incorporated RT-LAMP with CRISPR-Cas12a to allow detection of SARS-CoV-2 in respiratory swab RNA extracts in a colorimetric lateral flow assay (Broughton et al. 2020). Despite its capability to detect SARS-CoV-2 positive samples, it has reduced sensitivity when compare to qPCR assay performance in the same samples.

The present disclosure describes a method for detecting the presence of SARS-CoV-2 in a sample of DNA and RNA from nasal swab, plasma, serum, CSF, cell culture media, cellular suspensions, urine, blood, saliva, fecal, etc. Please refer to FIG. 1, which illustrates the method of this disclosure. In the first step, nucleic acids are extracted from the sample, such as a nasopharyngeal sample. The nucleic acid is then amplified targeting an SARS-CoV-2-specific sequence, if present. The product of amplification is then reacted with a gRNA-CRISPR system along with a reporter molecule. If the SARS-CoV-2-specific sequence is present, the gRNA will hybridize with it to activate the CRISPR effector protein, which then cleave the reporter molecule and one can determine the presence of SARS-CoV-2 based on measurement of signals produced by the reporter molecule.

Primers used in the DNA amplification step are designed to amplify only the SARS-CoV-2-specific gene sequences. For example, N gene, E gene or ORFlab gene of SRAS-CoV-2 has been identified and used for its detection.

The gRNA sequences were designed in accordance with the target fragments and the primers used in the DNA amplification step. In other words, the gRNA sequences are portions of the N gene or ORF lab gene. The target sequences and primers used are listed in Table 1.

TABLE 1
Oligonucleotide list

| | | |
|---|---|---|
| Name | Sequence | Target Gene |

For RT-PCR and RT-RPA

| | | |
|---|---|---|
| ORF1ab-F | CCCTGTGGGTTTTACACTTAA (SEQ ID NO. | ORF1ab |
| | 1) | |
| ORF1ab-R | ACGATTGTGCATCAGCTGA (SEQ ID NO. 2) | ORF1ab |
| N-F | GGGGAACTTCTCCTGCTAGAAT (SEQ ID | N-gene |
| | NO. 3) | |
| N-R | AGACATTTTGCTCTCAAGCTG (SEQ ID NO. | N-gene |
| | 4) | |
| RPP30-F | CTCGGATCCATCTCACTGCAA (SEQ ID NO. | RPP30 |
| | 5) | |
| RPP30-R | TGCAACAACATCATAGAGCCG (SEQ ID NO. | RPP30 |
| | 6) | |

For RT-LAMP*

| | | |
|---|---|---|
| F3-ORF1abF3-N | ATCCTAAAGGATTTTGTGACTT (SEQ ID NO. | ORF1abN- |
| | 7) | gene |
| B3-ORF1ab | TGCACTTACACCGCAAAC (SEQ ID NO. 8) | ORF lab |
| BIP-ORF1ab | TGTTTTTAAGTGTAAAACCACAGGAAGGT | ORF lab |
| | AAGTATGTACAAATACCTAC (SEQ ID NO. 9) | |
| FIP-ORF1ab | GTTATGGCTGTAGTTGTATCAACTGTTTAA | ORF lab |
| | AAACGATTGTGCATCA (SEQ ID NO. 10) | |
| F3-N | ACGTAGTCGCAACAGTTCAA (SEQ ID NO. 11) | N-gene |
| B3-N | TCTGCCGAAAGCTTGTGTT (SEQ ID NO. 12) | N-gene |
| BIP-N | ATCACCGCCATTGCCAGCCCAACTCCAGGC | N-gene |
| | AGCAGTAG (SEQ ID NO. 13) | |
| FIP-N | GCCAACAACAACAAGGCCAAACTAGTACG | N-gene |
| | TTTTTGCCGAGGC (SEQ ID NO. 14) | |

For CRISPR

| | | |
|---|---|---|
| gRNA-ORF1ab | UAAUUUCUACUCUUGUAGAUCACAUACC | ORF lab |
| | GCAGACGGUACAGAC (SEQ ID NO. 15) | |
| gRNA-N | UAAUUUCUACUCUUGUAGAUCUGCUGCU | N-gene |
| | UGACAGAUUGAAC (SEQ ID NO. 16) | |
| gRNA-RPP30 | UAAUUUCUACUCUUGUAGAUAGAGCAAC | RPP30 |
| | UUCUUCAAGGGCCC (SEQ ID NO. 17) | |
| Fluorescent reporter | FAM-TTTTTTTTTTTT-BHQ (SEQ ID NO. 18) | |

The present invention is exemplified with respect to N gene, E gene and ORFlab as the target fragment (RPP 30 was target as internal control gene). However, these targets are exemplary only, and the invention can be broadly applied to other regions of the SARS-CoV-2 genome. The following examples are intended to be illustrative only, and not unduly limit the scope of the appended claims.

### Materials and Method

1. Specimen Collection and Nucleic Acid Extraction

A total of 29 nasal swab specimens were collected based on clinical indications and current CDC guidance from Tulane Hospitals in New Orleans, La. from April 1 to Apr. 10, 2020. Subsequently, 100 μL of RNA were extracted from equal volumes of clinical sample using the QIAamp DSP Viral RNA Mini Kit, and extracted RNA was stored at −80° C. until analysis.

2. Amplification of Target Fragments

For RT-PCR reactions, 5 μL isolated RNA sample was mixed with 18 μL of one-step RT-PCR mix containing 10 μL of 2×Platinum™ SuperFi™ RT-PCR Master Mix (Thermo Fisher), 1 μL of forward primer (10 μM), 1 μL of reverse primer (10 μM), 0.2 μL of SuperScript™ IV RT Mix (Thermo Fisher), and 5.8 μL of nuclease-free water. Samples were then incubated in a T100 thermocycler (Bio-Rad, California) using a cDNA synthesis protocol (1 cycle at 55° C. for 10 min) immediately followed by a DNA amplification protocol (98° C. for 2 min; 35 cycles at 98° C. for 10 s, 60° C. for 10 s, and 72° C. for 15 s; followed by a final elongation step at 72° C. for 5 min). RPA pellets were resuspended in 29.5 μL of the supplied Rehydration Buffer, and 11.8 μL of this RPA solution, 0.5 μL of forward primer (10 μM), 0.5 μL of reverse primer (10 μM), 3.2 μL of nuclease-free water, 4 μL of MgOAC (280 mM), and 2 μL of the 2 μL of 120 μL isolated RNA sample were mixed and incubated at 42° C. for 20 min.

3. Optimization of CRISPR-Based Fluorescent Detection System

CRISPR-based fluorescent detection system (CRISPR-FDS) reactions were performed as follows: 20 μL of a sample RT-PCR or RPA reaction was transferred to a 96-well half-area plate and mixed with 10 μL of a CRISPR reaction mixture containing 3 μL of 10× NEBuffer™ 2.1, 3 μL of gRNA (300 nM), 1 μL of EnGen® Lba Cas12a (1 μM), 1.5 μL of fluorescent probe (10 μM), and 1.5 μL of nuclease-free water. After incubation at 37° C. for 20 minutes in the dark, fluorescence signal was detected using SpectraMax i3× Multi-Mode Microplate Reader (Molecular Devices, LLC., San Jose, USA).

For the Cas12a substrate-dependent kinetics study, the system was conducted with the molar ratio of Cas12a/gRNA to fluorescent probe at 1:5, 1:10, 1:15, 1:20, and 1:25. For the temperature-dependent kinetics study, reactions were performed using a 1:20 Cas12a/gRNA to fluorescent probe ratio at 27° C., 37° C., and 42° C. For target-dependent of kinetics study, the system was conducted with reactions were performed using a 1:20 Cas12a/gRNA to fluorescent probe ratio and 106, 107, 108, 109 and 1010 copies of the target fragments.

4. Sample Analysis

RT-qPCR was performed with the CDC 2019-Novel Coronavirus (2019-nCoV) Real Time RT-qPCR Diagnosis Panel. In these reactions, 5 μL of RNA sample was mixed with 1.5 μL of Combined Primer/Probe Mix, 5 μL of TaqPath™ 1-Step RT-qPCR Master Mix (4×), and 8.5 μL of nuclease-free water. RT-qPCR reactions were performed using a QuantStudio 6 Flex Real-Time PCR System (Thermo Fisher Scientific Inc., Waltham, USA) using the reaction conditions specified for this assay. For CRISPR-FDS assays, samples were processed as described above, using a 1:20 molar ratio of Cas12a/gRNA to fluorescent reporter and analyzed after incubation at 37° C. for 20 min.

5. CRISPR-FDS

Currently most of SARS-CoV-2 assays employ strategies that amplify species-specific regions of the SARS-CoV-2 RNA genome, including sites in the viral nucleocapsid (N) and envelope (E) genes, and open reading frame lab (ORF lab). The assay developed by the Chinese CDC targets sites with ORF lab and the N gene, while a test from the US CDC targets a site within the N gene, and the one developed by the World Health Organization targets a regions within the E gene, each of which contains a potential CRISPR recognition site (FIG. 1i). To compare the results from the instant disclosure with those from an establish test in clinical use, primers and gRNAs were designed (Table 1) to target the SARS-CoV-2 ORF lab and N regions analyzed by the Chinese CDC assay (FIG. 1). Bioinformatic analysis of these primers and gRNAs against common respiratory flora and other viral pathogens revealed that these sequences exhibited strong specificity for the SARS-CoV-2 genome. Sequence alignment of these SARS-CoV-2 target regions with corresponding sites in related beta coronaviruses that cause middle east respiratory syndrome (MERS-CoV), severe acute respiratory syndrome (SARS-CoV), and human coronavirus (Human-CoV) OC43/HKU1/229E/NL63, detected variable amounts of sequence variation between these species (FIG. 1C). The target region with the N gene exhibited the greatest degree of variation in this analysis, with multiple nucleotide differences detected along the aligned sequences. More differences were detected between SARS-CoV-2 and MERS-CoV than SARS-CoV in this region, in agreement with their phylogenetic distance. However, SARS-CoV still exhibited two or more variations with each N gene primer, while differed at three of four positions of the gRNA protospacer adjacent motif (PAM) required for CAS12a cleavage activity. The SARS-CoV ORF lab region differed from the matching gRNA at a single position outside its PAM, but both primer regions used to produce the target for this gRNA exhibited at least nucleotide variant, decreasing the likelihood for false positive SARS-CoV recognition events.

Analysis of RT-PCR-amplified and RT-RPA-amplified ORFlab target sequence from a SARS-CoV-2 RNA positive control sample demonstrated that both approaches produced strong signal relative to the background present in their matching negative control samples (FIG. 1C), and this difference was observed for both assay targets (FIG. 1D), and signal detected with MERS-CoV and SARS-CoV samples did not differ from negative control signal (FIG. 1E).

6. Covid-19 CRISPR-FDS Assay Optimization

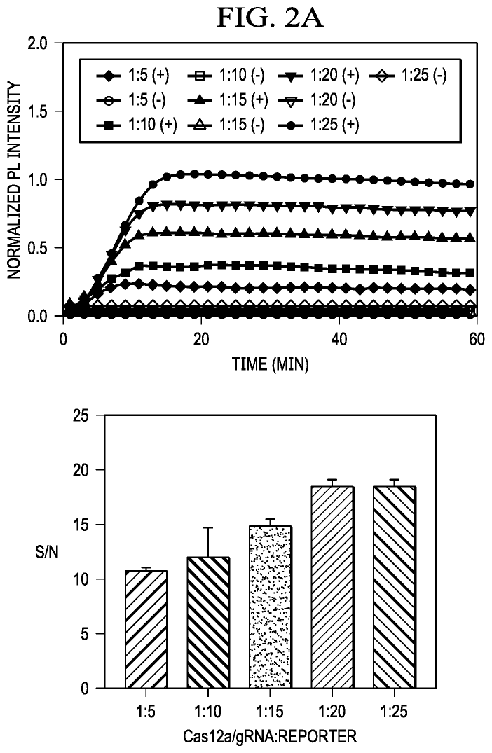

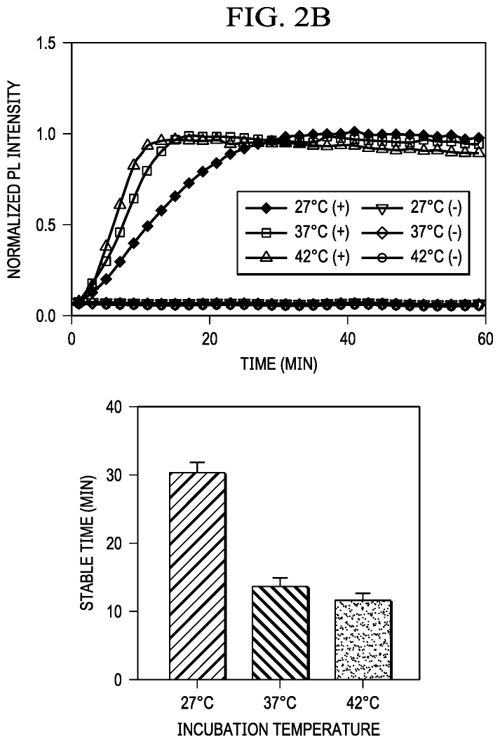

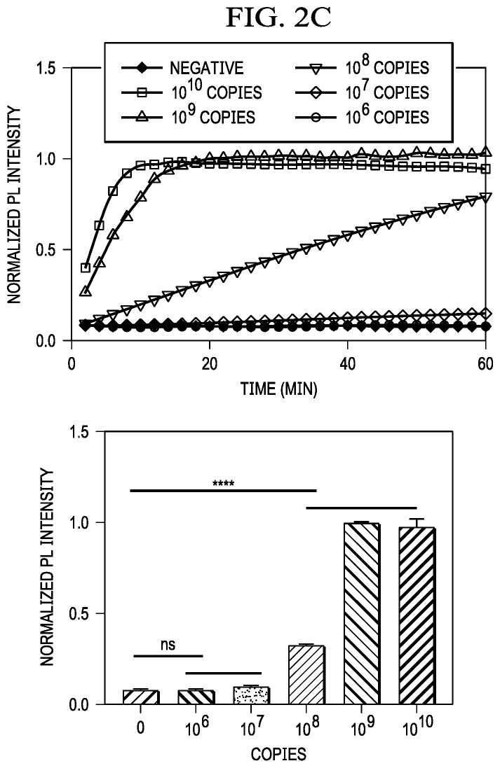

Since the CRISPR-based fluorescent reporter system determines the sensitivity of this assay, we conducted systematic studies of its reaction kinetics to optimize assay performance. CRISPR-mediated photoluminescence (PL) signal progressively increased with input fluorescent reporter substrate concentration, when CRISPR-FPR assays containing a constant amount of target RNA were incubated with increasing amounts of substrate (FIG. 2A). Signal-to-noise ratio increased with the ratio of reporter substrate to CRISPR/gRNA complex in this analysis, demonstrating the greatest signal-to-noise ratio at a 1:20 molar ratio of Cas12a/gRNA to reporter and plateauing or modestly decreasing at a 1:25 ratio, the highest analyzed in this study. A potential decrease detected at the highest reporter concentration could be due to the background fluorescent signal of un-cleaved reporter. The final CRISPR-FDS signal intensity did not differ with temperature in assays incubated at 27° C. to 42° C., but incubation temperature significantly altered the rate of substrate conversion, with reaction completion times decreasing from 30 minutes at 27° C., to 14 and 12 minutes at 37° C. and 42° C., respectively. CRISPR-FDS reactions can thus be performed at ambient temperature, or at elevated temperature using isothermal water baths or heat blocks, without influencing the final assay outcome.

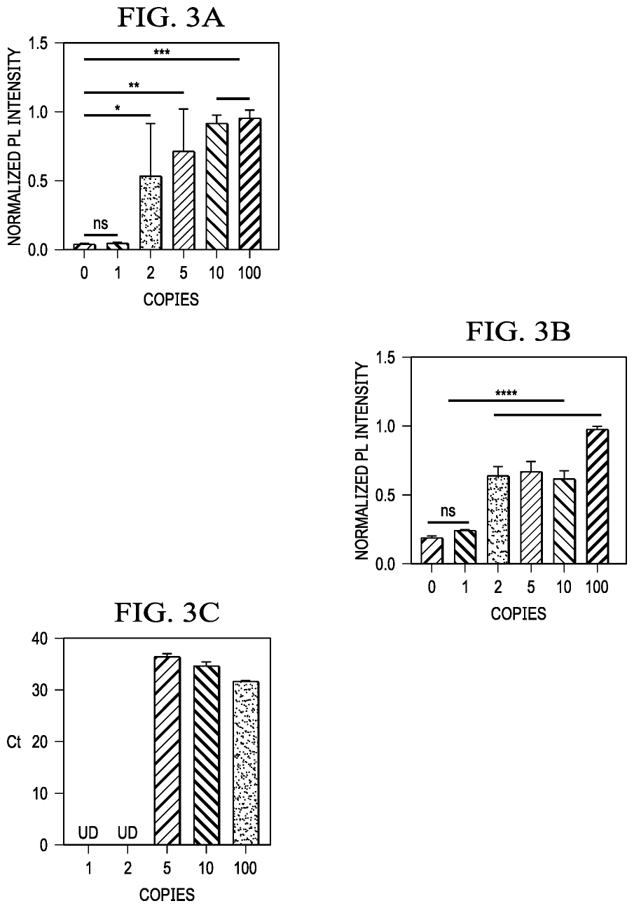

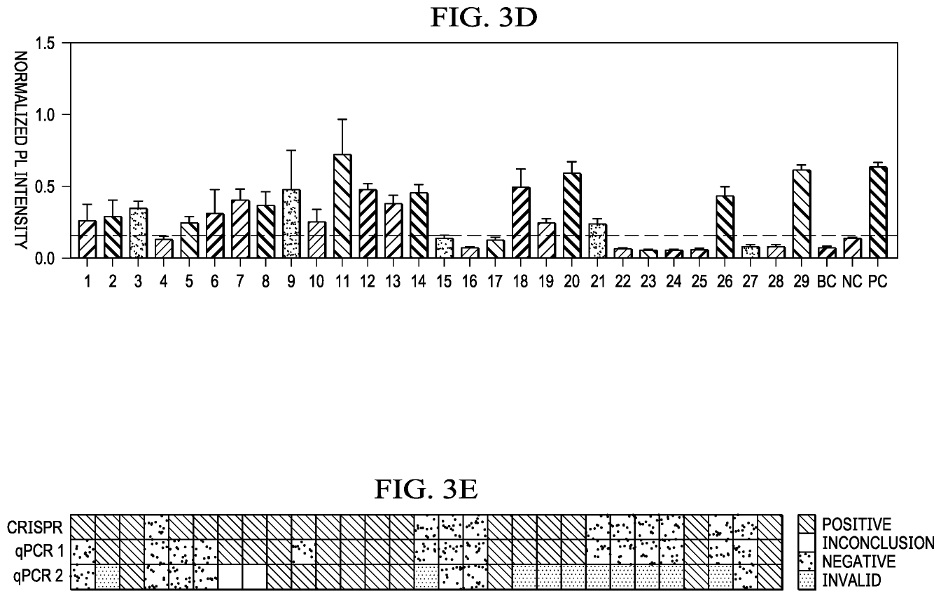

Cas12a/gRNA complex cleavage activity is dependent upon the concentration of amplified target present during the final assay incubation period, so that substrate conversion rates vary with the target amplicon concentration during the assay readout. Significant CRISPR-FDS signal was observed within a 20 min readout period only in assays spiked with 10′ copies of the target amplicon, while complete substrate conversion was detected only in samples spiked with >108 copies (FIG. 2C). The observed limit of detection (LOD) of 10′ amplicons per CRISPR-FDS readout sample indicates the toleration of this assay for RT-RPA or RT-PCR pre-amplification efficiency, since single copy cDNAs should be detected when amplification efficiencies are >0.69. COVID-19 CRISPR-FDS assays performed with RT-PCR and RT-RPA amplified samples showed that the assay could detect samples spiked with >2 copies of the target RNA sequence regardless of the method used in the pre-amplification step (FIG. 3(A)-(B)), in agreement with the calculated estimate for its LOD. This result compares favorably with the LOD of the qPCR gold-standard method, which was 5 copies/test (FIG. 3(C)). Signals of complete CRISPR-FDS reactions were also found to be stable for <1 hour at ambient temperature, reducing the need to read signal quickly when testing large sample batches.

COVID-19 CRISPR-FDS Diagnostic Performance

For the analysis of clinical samples, a COVID-19 CRISPR-FDS assay result was considered positive if it was equal or greater than a cut-off threshold equal to the mean signal of the negative control samples plus three times its standard deviation. Using this criterion, 19 of 29 nasal swab samples obtained in Tulane Hospital New Orleans, La. were found to be SARS-CoV-2 positive (FIG. 3(D)). These results demonstrated good overall agreement with valid and conclusive test results from the state and hospital laboratories that were generated using a CDC-authorized qPCR method (FIG. 3(E)). However, the COVID-19 CRISPR-FDS assay detected SARS-CoV-2 signal in three samples that were identified as negative samples by the state and hospital laboratories (Samples 1, 5 and 6). In the absence of serology data or other information, it is not clear if these three samples represent false positive CRISPR-FDS assay results, or if they represent positive samples that were missed by the qPCR method.

Although qRT-PCR is the most widely used diagnostic method for COVID-19, its sensitivity has not proven satisfactory, resulting in a relatively high number of false-negative results so that a substantial number of infected individuals do not receive proper diagnoses and treatment. In a study of more than 1,000 patients, 75% COVID-19 suspects had negative qRT-PCR test results but positive chest CT results, and 48% of these patients were considered highly likely to have COVID-19, with an additional 33% considered probable cases. Notably, there is a high frequency of invalid or inconclusive test results from samples analyzed in a clinical laboratory, all of which gave valid results when analyzed by RT-PCR in the state testing laboratory or by our CRISPR-FDS assay.

Many CRISPR protocols use paper strips to detect the signal output. This is a good solution for single sample testing since it does not require any equipment to read the results, but it is not suitable for high-throughput screening necessary in clinical settings and has lower sensitivity than fluorescence-based detection methods. The present disclosure CRISPR-FDS assay can be readily performed in 96-well microtiter plates and read with fluorescent plate readers found in most clinical laboratories to allow sensitive and high-throughput SARS-CoV-2 detection. Finally, the results indicate that our CRISPR-FDS demonstrates results comparable to those obtained with a CDC-approved qPCR assay in a state testing lab, but produced more valid results than were obtained when the same qPCR assay was employed in a clinical setting. CRISPR-FDS thus produces sensitive and robust results using readily available equipment and streamlined, high-throughput workflow suitable for use in clinical laboratories, and potentially applicable to point of care settings with the appropriate equipment.

Smartphone-Based RNA Extraction-Free Saliva Assay Using CRISPR-FDS

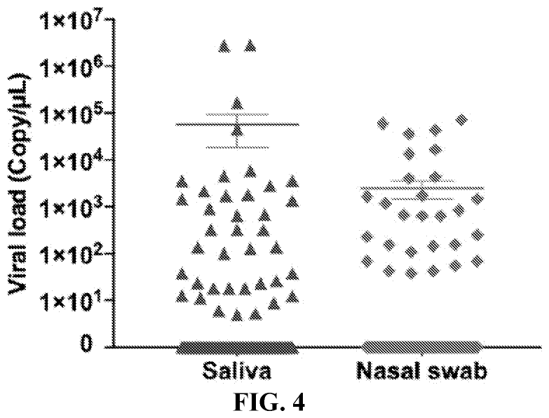

An alternative detection method as described herein is based on saliva sample instead of nasal swap. Recent studies indicate that saliva and nasopharyngeal SARS-CoV-2 results exhibit correlation during early infection, and development of saliva-based COVID-19 assays could reduce or eliminate the involvement of medical personnel in sample collection, since saliva collection would not require special materials, training or infra-structure. In one experiment, CRISPR-FDS analysis of 103 paired saliva and nasal swab samples obtained from individuals screened for COVID-19 detected SARS-CoV-2 RNA in more saliva than nasal swab samples, as shown in FIG. 4.

Conventionally, most high sensitivity NAA assays analyze purified RNA samples isolated in multi-step procedures that require additional laboratory equipment. However, this RNA-isolation step may not be practical when an on-site analysis is required without the necessary equipment to do so. Therefore, an alternative viral lysis procedure was developed that would allow viral lysis samples to be directly analyzed by CRISPR-FDS without a separate isolation step, using a cell lysis procedure compatible with PCR as the base condition. There are several variables in the method, including the saliva sample-to-lysis buffer ratio, the temperature and duration of denaturing the RNA.

The assay is performed as described below.

QuickExtract DNA Extraction Solution (Lucigen) was mixed with saliva samples as indicated to release viral RNA, since this solution is compartible with PCR, RPA and CRISPR reactions. Saliva and lysis buffer mixtures were then incubated at a predetermined temperature for a predetermined duration, after which 5 μL of the lysed sample was mixed with a RT-RPA solution.

The RT-RPA solution was prepared by suspending recombinase polymerase amplification (RPA) pellets from the TwistAmp® Basic kit (ABAS03KIT; TwistDx Limited; Maidenhead, UK) in 29.5 μL of the supplied Rehydration Buffer, and 11.8 μL of this RPA solution, 0.5 μL of forward primer (10 μM), 0.5 μL of reverse primer (10 μM), 3.2 μL of nuclease-free water, 4 μL of magnesium acetate (MgOAc; 280 mM), 1 μL SuperScript IV reverse tran-scriptase and 5 μL of the lysed sample were mixed and incubated at 42° C. for 20 min.

CRISPR reaction mixture as used herein comprises 3 μL of 10× NEBuffer 2.1, 3 μL of gRNA (300 nM), 1 μL of EnGen® Lba Cas12a (1 μM), 1.5 μL of fluorescent probe (10 μM), and 1.5 μL of nuclease-free water. After incubation with the RT-RPA mixture at 37° C. for 20 min in the dark, fluorescence signal was detected as further described below using the fluorescence assay reader.

The sample-to-lysis buffer ratio may vary depending on the conditions, as higher sample-to-lysis buffer ratio may increase the effectiveness but may not always be practical. In one embodiment, the sample-to-lysis buffer ratio ranges from 1:1 to 1:10.

For lysis/RT-RPA reaction temperature, it can range from 37° C. to 95° C. In one embodiment, the lysis and RT-RPA is performed at 37° C. for easier point-of-care consideration.

The duration to perform lysis and RT-RPA may vary based on the sample-to-buffer ratio or the temperature under which the lysis/RT-RPA is performed. In one embodiment, the duration can range from 1 to 30 minutes. In one embodiment of 1:1 sample-to-buffer ratio at 37° C., the duration is 10 minutes.

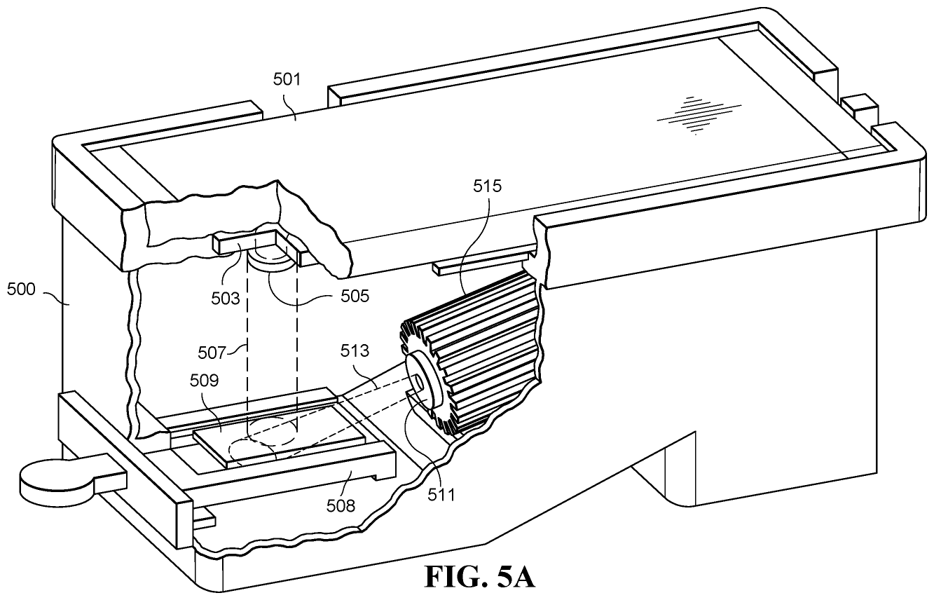

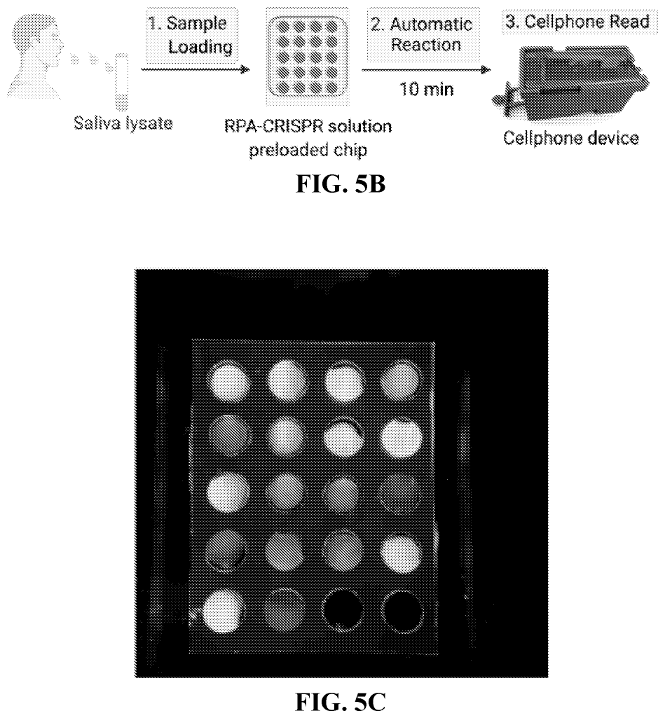

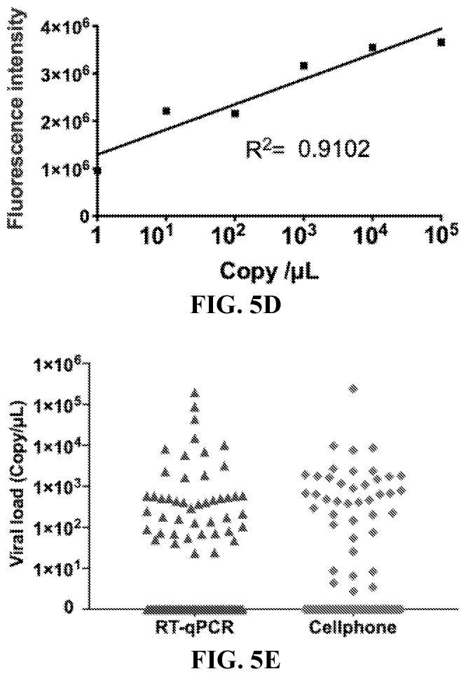

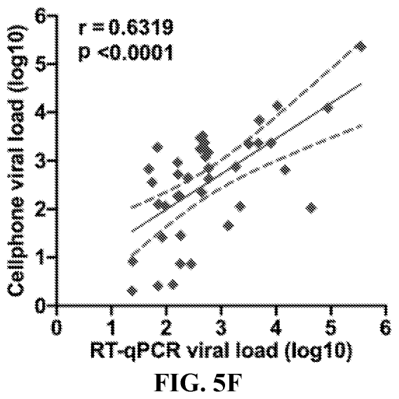

Particularly, the fluorescent detection may be performed using a cellphone and a fluorescent assay reader, as shown in FIG. 5A. The fluorescent assay reader can be 3-D printed to suit different needs. The fluorescent assay reader 500 has a seat 508 for sitting a reaction chip 509. The ass has a laser diode 511 that can emit excitation light 513 to a reaction chip 509. The fluorescent assay reader 500 further has a smartphone holder 502 that holds a smartphone 501 above the reaction chip 509. A fluorescence filter 503 and an external lens 505 are provided between the camera of the smartphone 501 and the reaction chip 509, such that the camera of the smart phone 501 can capture fluorescent readings from the reaction chip 509 using light 507 from the smartphone. Optionally heat sink 515 is provided around the laser diode 511 to dissipate heat.

It is noted that the fluorescent assay reader can have different designs or configurations based on a particular need. The fluorescent assay reader should comprise at least a phone holder, lenses, laser diode, chip adaptor, fluorescent filter, and temperature control modules to perform necessary detection.

In one embodiment, the fluorescent assay reader further comprises the capability to control the image capturing and temperature modulation through a connection with a smartphone app. For example, the fluorescent assay reader may establish a wired or wireless connection with a smartphone, including but not limited to WiFi, Bluetooth, close range wireless communication. The user then can control the temperature as need, the duration of that change, fluid flow, as well as capturing the fluorescent image through the smartphone app.

In one embodiment, the fluorescent assay reader comprises multiple laser sources with paired filter integration to detect multiple targets, as well as multiple channels of fluorescent signals.

In one embodiment, the fluorescent assay reader is capable of detecting chip loading status. For example, the fluorescent assay reader may use visual or micro valve actuation to detect whether the reaction chip has been loaded, as well as the progress of the detection process.

In one embodiment, the smartphone holder is configured such that the width and length thereof can be adjusted to fit various smartphone sizes.

In one embodiment, the fluorescent assay reader may comprise a control screen, providing the user visual and touch-screen control of the detection process, including loading samples, controlling microfluidic flow, varying temperature and duration, emitting laser at various angle and adjusting power thereof, switching filter, calibrating camera location and heating module location, as well as capturing images.

In one embodiment, the smartphone app provides functions of controlling the fluorescent assay reader, including but not limited to: capture image, reaction time, reaction temperature, laser and filter switch, laser power; calibration of camera location, heating module location, laser location. In one embodiment, the smartphone app further provides sample analysis functions, including but not limited to: distinguishing positive and negative results based on fluorescent intensity, annotating sample, and quantitative calculation. Analysis of mutation, multiple disease. In one embodiment, the smartphone app further provides reporting functions, including but not limited to: providing comprehensive result of SARS-CoV-2 infection (positive/negative, wild type/mutation), uploading results to cloud for health provider, CDC, local health department; exporting results in multiple formats (pain text, PDF, image etc.); and exporting with encryption. In one embodiment, the smartphone app is capable of connecting to an insurance account to communicate the testing results.

The reaction chip may have different configuration, as long as it has the necessary structure to hold a sufficient amount of saliva sample and lysis/RT-RPA buffer. In one embodiment, the reaction chip is capable of integrating sample isolation, amplification and detection all in one chip. For example, the reaction chip may comprise different zones each dedicated for sample isolation, nucleic acid amplification, and detection, with microfluidic channels interconnecting each zone to streamline the process.

The reaction chip can also be used for multiple sample detection. This includes detecting different samples, different disease targets, or different genotype/mutations of the same pathogen. For example, the reagents pre-loaded in the wells can be the same for each well to detect samples from different subjects; or alternatively, the reagents pre-loaded in the wells can be used to detect different pathogens such as SARS-CoV-2, pneumonia, or other pathogens of interest; or alternatively, the reagents pre-loaded in the wells can be used to detect different mutations or genotypes, such as primers/gRNAs targeting variants of SARS-CoV-2.

For easier handling and sanitation reasons, the reaction chips are designed such that the wells for isolation/amplification/detection are covered to prevent any spillage, especially when loaded with samples. The reaction chip can also be readily disposable from the fluorescent assay reader once the detection step is completed.

In one embodiment, the reaction chip has a dimension of 25×35×4 mm that is suitable for an on-chip CRISPR-FDS saliva assay inserted into the smartphone fluorescent assay reader described above. The reaction chip comprises a layer of polydimethylsiloxane (PDMS) mounted on a glass microscope slide. PDMS was selected for this application because it is a chemically inert and optically clear silicone elastomer that can spontaneously adhere to glass surfaces after plasmonic oxidation, therefore allowing wells to be excited by low-angle laser illumination. Additionally, PDMS/glass format can be readily manufactured and modified in a cost-effective way.

In this embodiment, the reaction chip comprises five reaction wells (ID.=3.5 mm, maximum volume≈28 μL) to allow the analysis of five assays in parallel (e.g., three test wells, one PC well and one NC well), where wells were designed to contain sufficient volume for sensitive detection. Reaction wells were arranged in a pentagonal array illuminated by a laser diffused to cover the ˜20×20 mm field of view of the smartphone camera. A pentagonal array was chosen to minimize illumination differences in this embodiment, however, more compact arrays containing more wells could be employed to simultaneously analyze samples from multiple individuals and/or to accommodate a standard curve to quantify viral load.

Alternative designs could also employ microfluidic channels to load multiple wells from a single inlet port and employ film to seal the chip after sample loading to prevent environmental contamination by assay amplicons. To verify the utility of this chip design, we employed an on-chip CRISPR-FDS assay to analyze saliva from 12 COVID-19 patients and 6 healthy controls. The results (not shown) captured and analyzed by the fluorescent microplate reader distinguished saliva from patients with positive and negative nasal RT-qPCR results, supporting the feasibility of the on-chip method for diagnosing COVID-19.

This integrated system was designed to utilize a method of detecting SARS-CoV-2 from saliva samples as shown in FIG. 5B. In one embodiment, a typical saliva volume of 0.5-3 mL is collected in a tube prefilled with 3 mL of lysis buffer, which is then capped and heated at >37° C. for >5 min, after which approximately 5 μL of the lysed sample is added to each sample well of an assay chip containing 10 μL/well of pre-mixed RPA and CRISPR solution. This chip is then incubated for >10 min at room temperature and then inserted into the smartphone reader, the laser diode is turned on, and assay chip images are captured by the smartphone camera.

FIG. 5C shows an exemplary image that shows the fluorescent reading using the method and reaction chip with the fluorescent assay reader as described above. The field of view (FOV) of this device was increased by adding an external lens with a 50 mm focal length. This yielded a FOV compatible with diameter of the reaction well array on the reaction chip without producing significant aberration. This device also employed a 100 mW laser diode with a high incidence angle to allow sensitive detection of reaction products while minimizing background noise. A 525 nm filter was used with a smartphone to take the picture.

FIG. 5D shows the standard curve of the on-chip CRISPR-FDS saliva test read by the smartphone device. Here the analytical performance of this fluorescent assay reader was examined by analyzing on-chip assays of a SARS-CoV-2 RNA concentration curve generated by serially diluting heat-inactivated SARS-CoV-2 virus in healthy donor saliva. This standard curve demonstrated good linearity (R2=0.91) over a broad viral concentration range (1-105 copies/μL) and a calculated LOD of 0.38 copy/μL when read on the smartphone device.

In FIG. 5E, both CRISPR-FDS and RT-qPCR were used to blindly analyze 103 saliva samples from individuals screened for COVID-19. Results show that the CRISPR-FDS plate reader and smartphone assays and the standard RT-qPCR as-say detected similar numbers of SARS-CoV-2 positive saliva samples.

In an analysis using RT-qPCR as the reference standard, CRISPR smartphone results exhibited a 1.3% false positive rate with saliva but complete concordance with RT-qPCR results for swab samples, while CRISPR plate reader results perfectly matched RT-qPCR saliva results, but exhibited a 2.3% false negative rate with nasal swab samples.

Viral load was strongly correlated in the 43 saliva samples that tested positive by both the on-chip smartphone assay and conventional RT-PCR analysis, as shown in FIG. 5F, and exhibited similar mean values (3803 versus 1797 copies/μL).

These results demonstrate that the saliva-based on-chip CRISPR-FDS assay for COVID-19 exhibits complete concordance with RT-qPCR when analyzing saliva samples spiked with SARS-CoV-2 concentrations that fall within the linear range of the RT-PCR assay, an estimated 0.38 copies/μL LOD, and a broad linear range (1-105 copies/μL). Notably, this on-chip assay does not require RNA isolation but exhibits a LOD similar to RT-qPCR (0.38 copies/μL versus 1 copy/μL) and greater than CRISPR-based COVID-19 assays proposed for point-of-care diagnosis (4-10 copies/μL), all of which require separate RNA isolation procedures.

This assay platform has several features that should render it suitable for use in a variety of point-of-care testing environments. First of all, it analyzes saliva samples that can be collected by the subject being tested to reduce the demands upon medical personnel. Secondly, it exhibits robust performance in response to large variations in sample dilution and denaturation and CRISPR-FDS reaction temperatures and times. Lastly, it utilizes an inexpensive and highly portable smartphone-based reader, which could also speed and simplify coded data reporting from remote testing sites.

Notably, the estimated sensitivity of the smartphone-based device for SARS-CoV-2 approaches the sensitivity detected in off-chip assays read by a fluorescent microplate reader (0.38 versus 0.05 copies/μL), supporting the potential for broad use of this platform in screening and diagnosis. This sensitivity was achieved by low incident angle illumination of the assay chip by a 100 mW laser diode powered by AAA batteries that achieved high excitation intensity and signal-to-noise conditions for assay well image capture. Sample focusing and image acquisition were achieved by the built-in smartphone camera app, eliminating the need for the mechanical focusing, and thus reducing weight and cost while enhancing the optical stability and user-friendliness of the device.

In one embodiment, the microfluidic reaction chip can regulate the flow and mixing of reaction samples, with heating elements that can be controlled by the smartphone to precisely regulate reaction temperatures. In one embodiment, the reaction chip can also have barcodes to facilitate data reporting. In one embodiment, a customized smartphone application can be used to regulate different reaction zones on the chip, for example lysis, RT-RPA and CRISPR-FDS reaction, followed by automatic capture images of the assay wells with the smartphone camera. The app can also analyze the data and remotely report the assay data to a server to support telehealth efforts, and possibly aggregating data to governmental organizations tasked with making public health decisions.

The following references are incorporated by reference in their entirety for all purposes.
Broughton, J. P., Deng, X., Yu, G., Fasching, C. L., Servellita, V., Singh, J., Miao, X., Streithorst, J. A., Granados, A., Sotomayor-Gonzalez, A., Zorn, K., Gopez, A., Hsu, E., Gu, W., Miller, S., Pan, C.-Y., Guevara, H., Wadford, D. A., Chen, J. S., Chiu, C. Y., 2020. CRISPR-Cas12-based detection of SARS-CoV-2. Nature Biotechnology.
Chen, J. S., Ma, E., Harrington, L. B., Da Costa, M., Tian, X., Palefsky, J. M., Doudna, J. A., 2018. CRISPR-Cas12a target binding unleashes indiscriminate single-stranded DNase activity. Science 360(6387), 436-439.
Gootenberg, J. S., Abudayyeh, O. O., Lee, J. W., Essletzbichler, P., Dy, A. J., Joung, J., Verdine, V., Donghia, N., Daringer, N. M., Freije, C. A., Myhrvold, C., Bhattacharyya, R. P., Livny, J., Regev, A., Koonin, E. V., Hung, D. T., Sabeti, P. C., Collins, J. J., Zhang, F., 2017. Nucleic acid detection with CRISPR-Cas13a/C2c2. Science 356(6336), 438-442.
Li, S. Y., Cheng, Q. X., Wang, J. M., Li, X. Y., Zhang, Z. L., Gao, S., Cao, R. B., Zhao, G. P., Wang, J., 2018. CRISPR-Cas12a-assisted nucleic acid detection. Cell Discov 4, 20.
Lucia, C., Federico, P.-B., Alejandra, G. C., 2020. An ultrasensitive, rapid, and portable coronavirus SARS-CoV-2 sequence detection method based on CRISPR-Cas12. bioRxiv
Pardee, K., Green, A. A., Takahashi, M. K., Braff, D., Lambert, G., Lee, J. W., Ferrante, T., Ma, D., Donghia, N., Fan, M., Daringer, N. M., Bosch, I., Dudley, D. M., O'Connor, D. H., Gehrke, L., Collins, J. J., 2016. Rapid, Low-Cost Detection of Zika Virus Using Programmable Biomolecular Components. Cell 165(5), 1255-1266.

What is claimed is:

1. A method of detecting SARS-CoV-2 in a sample, comprising the steps of:
a) optionally extracting RNAs from a sample;
b) reverse-transcribing the RNAs into a mixture of DNAs;
c) amplifying at least one SARS-CoV-2-specific target DNA sequence from the mixture of DNAs; and
d) detecting presence of the SARS-CoV-2-specific target nucleic acid sequence using a CRISPR-mediated system;
wherein said CRISPR-mediated system comprises a CRISPR effector protein, a guide RNA (gRNA) that hybridizes with the SARS-CoV-2-specific target nucleic acid fragment, and a reporter molecule that is detectable on cleavage by said CRISPR effector protein.

2. The method of claim 1, wherein in step a) the extracting step further comprising:
a-1) depleting human RNA using anti-human RNA antibodies.

3. The method of claim 1, wherein in step a) the extracting step further comprising:
a-2) enriching SARS-CoV-2 RNAs by using anti-SARS-CoV-2 RNA antibodies.

4. The method of claim 1, wherein step c) is carried out using polymerase chain reaction (PCR), recombinase polymerase amplification (RPA), nucleic acid sequence-based amplification (NASBA), rolling circle amplification (RCA), or loop-mediated isothermal amplification (LAMP).

5. The method of claim 4, wherein step c) is carried out using PCR or RPA.

6. The method of claim 1, wherein in step d) the CRISPR effector protein is selected from a group consisting of Cas12a, Cas9 and Cas13.

7. The method of claim 1, wherein the reporter molecule is a single-stranded DNA or a single-stranded RNA labeled with fluorescence and quencher, gold nanoparticles, or biotin-FAM.

8. The method of claim 7, wherein the reporter molecule is 5′-6-FAM-TTTTTTTTTTTT-BHQ1 (SEQ ID NO. 18).

9. The method of claim 1, wherein said sample is obtained from nasopharyngeal swap, oropharyngeal swab, nasopharyngeal wash, nasopharyngeal aspirate, nasal aspirate, nasal mid-turbinate swab, bronchoalveolar lavage, tracheal aspirate, pleural fluid, lung biopsy, sputum, or saliva.

10. The method of claim 1, wherein in steps b) and c) are carried out in a temperature between 32° C. to 45° C.

11. The method of claim 1, wherein the target DNA sequence is a portion of N gene, E gene, M gene, S gene or ORF lab gene from the SARS-CoV-2 genome.

12. The method of claim 1, wherein in step c) more than one target DNA sequences are amplified.

13. The method of claim 1, wherein a pair of primers are used in step c) for the DNA amplification, wherein the primers are SEQ ID NOs. 1 & 2 or SEQ ID NOs. 3 & 4 or SEQ ID NOs. 5 & 6.

14. The method of claim 1, wherein primers are used in step c) for the DNA amplification, wherein the primers are SEQ ID NOs. 7&8&9&10 or SEQ ID NOs. 11&12&13&14.

15. The method of claim 1, wherein in step d) the gRNA has at least one of the following sequences: SEQ ID NOs. 15 and 17.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. A method of detecting the presence of a pathogen in a sample, comprising the steps of:
a) optionally extracting RNAs from the sample;
b) reverse-transcribing said RNAs into a DNA mixture;
c) amplifying a target DNA sequence from said DNA mixture or from the extracted RNAs in step a), wherein a pair of primers matching a portion of said target DNA are used; and
d) detecting presence of the target DNA fragment in said amplified DNA using a CRISPR-mediated system;
wherein said CRISPR-mediated system comprises Cas12a, a guide RNA (gRNA), and a reporter molecule that is detectable on cleavage by Cas12a, and wherein said gRNA matches a portion of said target DNA; and
wherein said pathogen is selected from a group consisting of: human coronavirus 229E, human coronavirus OC43, human coronavirus HKUJ, human corona virus NL63, SARS-coronavirus, MERS-coronavirus, Adenovirus, Human Metapneumovirus (hMPV), Parainfluenza virus 1-4, Influenza A & B, Enterovirus, Respiratory syncytial virus, Rhinovirus, Chlamydia pneumoniae, Haemophilus influenzae, Legionella pneumophila, Mycobacterium tuberculosis, Streptococcus pneumoniae, Streptococcus pyogenes, Bordetella pertussis, Mycoplasma pneumoniae, Pneumocystis jirovecii (PJP), Candida albicans, Pseudomonas aeruginosa, Staphylococcus epidermis, Staphylococcus salivarius.

28. A fluorescent assay device, comprising:
a) a phone holder;
b) a lens;
c) a fluorescence filter;
d) an adaptor to receive a reaction chip;
e) a light source, wherein the light source emits a light to the reaction chip to excite fluorescence signals; and
f) a temperature control module capable of controlling temperatures on the reaction chip;
wherein the phone holder receives a smartphone having a camera, and the lens and the fluorescence filter are aligned between the camera and the reaction chip.

29. The fluorescent assay device of claim 28, further comprising: g) a controller, wherein the controller controls the light source and the temperature control module.

30. The fluorescent assay device of claim 29, wherein the reaction chip comprises a plurality of wells for receiving reagents and samples.

31. The fluorescent assay device of claim 30, wherein the plurality of wells are connected through microfluidic channels.

32. The fluorescent assay device of claim 31, wherein the controller controls fluid flow within the microfluidic channels between the plurality of wells.

33. The fluorescent assay device of claim 29, further comprising: i) a communication module operatively connected to the controller, wherein the communication module establishes a communication with a smartphone, wherein the communication is wired or wireless.

34. The fluorescent assay device of claim 33, wherein the smartphone controls the fluorescent assay device through the communication.

35. The fluorescent assay device of claim 34, wherein the smartphone controls the light source, the temperature control module through the communication with the controller, and the camera.

36. The fluorescent assay device of claim 35, wherein the smartphone analyzes images captured by the camera.

37. The fluorescent assay device of claim 28, wherein the phone holder is adjustable in size.

38. The fluorescent assay device of claim 28, wherein the fluorescent assay device further comprises a battery power source.
